(19) United States
(12) Patent Application Publication
(10) Pub. No.: US 2005/0096308 A1
(43) Pub. Date: May 5, 2005

(54) Antibacterial agents

(76) Inventors: Edmund Lee Ellsworth, Pinckney, MI (US); Richard J. Sciotti, Saline, MI (US); Jeremy Tyson Starr, Ann Arbor, MI (US)

Correspondence Address: WARNER-LAMBERT COMPANY, 2800 PLYMOUTH RD, ANN ARBOR, MI, 48105, US

(21) Appl. No.: 10/936,222

(22) Filed: Sep. 8, 2004

Related U.S. Application Data
(60) Provisional application No. 60/502,305, filed on Sep. 12, 2003.

(51) Int. Cl. A61K 31/517; C07D 43/02; A61K 31/675

(52) U.S. Cl. 514/210.21; 544/284; 514/81; 544/244

(57) ABSTRACT

Compounds of formula I and methods for their preparation are disclosed. Further disclosed are methods of making biologically active compounds of formula I as well as pharmaceutically acceptable compositions comprising compounds of formula I. Compounds of formula I as disclosed herein can be used in a variety of applications including use as antibacterial agents.

This application claims benefits of U.S. Provisional Application No. 60/502,305, filed on Sep. 12, 2003.

### FIELD OF THE INVENTION

The invention relates to compounds bearing an aminoquinazolinedione core structure which exhibit antibacterial activity, methods for their preparation, as well as pharmaceutically acceptable compositions comprising such compounds.

### BACKGROUND OF THE INVENTION

Antibacterial resistance is a global clinical and public health problem that has emerged with alarming rapidity in recent years and undoubtedly will increase in the near future. Resistance is a problem in the community as well as in health care settings, where transmission of bacteria is greatly amplified. Because multiple drug resistance is a growing problem, physicians are now confronted with infections for which there is no effective therapy. The morbidity, mortality, and financial costs of such infections pose an increasing burden for health care systems worldwide. Strategies to address these issues emphasize enhanced surveillance of drug resistance, increased monitoring and improved usage of antimicrobial drugs, professional and public education, development of new drugs, and assessment of alternative therapeutic modalities.

As a result, alternative and improved agents are needed for the treatment of bacterial infections, particularly for the treatment of infections caused by resistant strains of bacteria, e.g. penicillin-resistant, methicillin-resistant, ciprofloxacin-resistant, and/or vancomycin-resistant strains.

### SUMMARY OF THE INVENTION

These and other needs are met by the present invention, which is directed to a compound of formula I 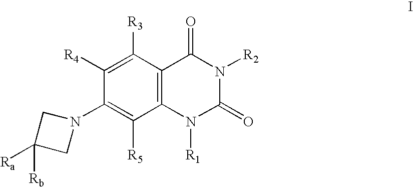
or a pharmaceutically acceptable salt thereof wherein
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is H,
   NH₂, 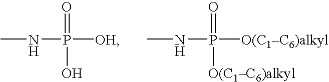
   NH(C₁-C₆)alkyl,
   NH(C₃-C₆)cycloalkyl,
   NH-heteroaryl,
   NHSO₂—(C₁-C₆)alkyl,
   NHSO₂-aryl,
   NHSO₂-heteroaryl, 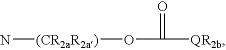
   wherein, Q is O or is absent, and R₂ₐ and R₂ₐ are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 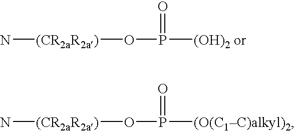
   wherein R₂ₐ and R_{2a′} are as defined above, 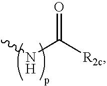
   wherein 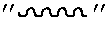
   indicates the point of attachment, p is 0 or 1, and
      R_{2c} is H,
         (C₁-C₆)alkyl,
         (C₃-C₇)cycloalkyl,
         aryl,
         heterocyclo,
         heteroaryl, or 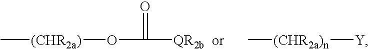
         wherein R₂ₐ, R_{2b}, and Q, are as defined above, n is an integer from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 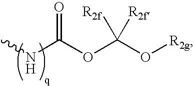
   wherein q is 0 or 1, R_{2f} and R_{2f} are each independently H, (C₁-C₆)alkyl, aryl, or heteroaryl, or taken together with the carbon to which they are attached form a 3, 4, 5, or 6 membered ring, and R_{2g} is
      (C₁-C₆)alkyl,
      (C₃-C₇)cycloalkyl,
      aryl, or
      heterocyclo, or
      heteroaryl;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
Rₐ is H,
   aryl,
   (C₁-C₆)alkyl,
   halo(C₁-C₆)alkyl,
   (C₃-C₆)cycloalkyl, 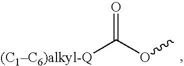
   wherein 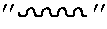
   indicates the point of attachment and Q is O or is absent, 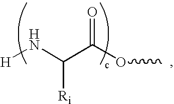
   wherein 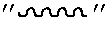
   indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 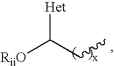
   wherein 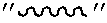
   indicates the point of attachment, het is a 5- or 6-membered heterocyclo or heteroaryl group, and x is an integer of from 0 to 10; 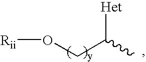
   wherein 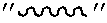
   indicates the point of attachment, het is as defined above, and y is an integer of from 1 to 10;
   wherein Rᵢᵢ is H,
      (C₁-C₆)alkyl,
      PO(OH)₂,
      PO((C₁-C₆)alkyl)₂, 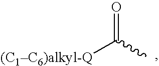
      as defined above, or 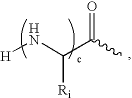
      as defined above;
   provided that 3 or fewer of R_{c} R_{d}, Rₑ, and R_{f} are H; or
R_{b} is OH,
   OPO(OH)₂,
   OPO((C₁-C₆)alkyl)₂ 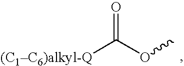
   wherein 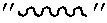
   indicates the point of attachment and Q is O or is absent, 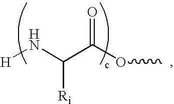
   wherein 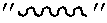
   indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 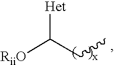
   wherein 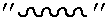
   indicates the point of attachment, het is a 5- or 6-membered heterocyclo or heteroaryl group, and x is an integer of from 0 to 10; 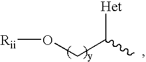
   wherein 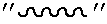
   indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, 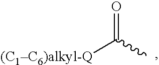
         as defined above, or 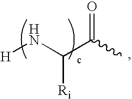
         as defined above.

What is also provided is a compound which is:

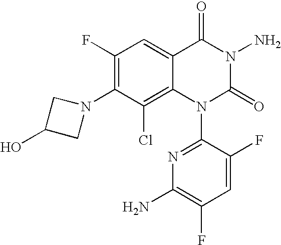

### 3-Amino-1-(6-amino-3,5-difluoro-pyridin-2-yl)-8-chloro-6-fluoro-7-(3-hydroxy-azetidin-1-yl)-1H-quinazoline-2,4-dione

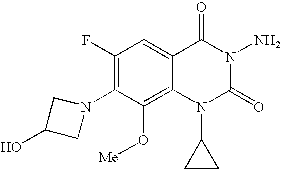

### 3-Amino-1-cyclopropyl-6-fluoro-7-(3-hydroxy-azetidin-1-yl)-8-methoxy-1H-quinazoline-2,4-dione

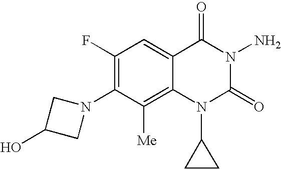

### 1-Cyclopropyl-6-fluoro-7-(3-hydroxy-azetidin-1-yl)-8-methyl-4-oxo-1,4-dihydro-quinoline-3-carboxylic Acid

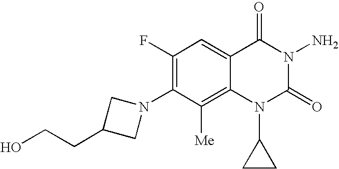

### 3-Amino-1-cyclopropyl-6-fluoro-7-[3-(2-hydroxy-ethyl)-azetidin-1-yl]-8-methyl-1H-quinoline-2,4-dione

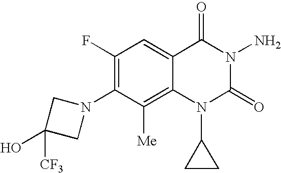

### 3-Amino-1-cyclopropyl-6-fluoro-7-(3-hydroxy-3-trifluoromethyl-azetidin-1-yl)-8-methyl-1H-quinazoline-2,4-dione;

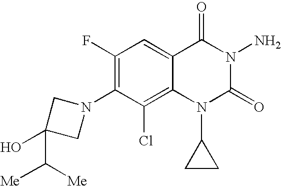

### 3-Amino-8-chloro-1-cyclopropyl-6-fluoro-7-(3-hydroxy-3-isopropyl-azetidin-1-yl)-1H-quinazoline-2,4;dione

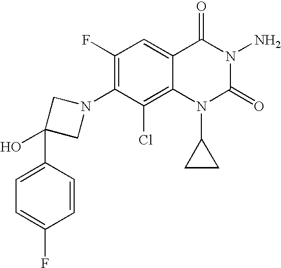

### 3-Amino-8-chloro-1-cyclopropyl-6-fluoro-7-[3-(4-fluoro-phenyl)-3-hydroxy-azetidin-1-yl]-1H-quinazoline-2,4-dione; or

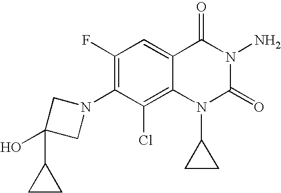

### 3-Amino-8-chloro-1-cyclopropyl-7-(3-cyclopropyl-3-hydroxy-azetidin-1-yl)-6-fluoro-1H-quinazoline-2,4-dione

What is also provided is a pharmaceutical formulation comprising a compound of one of formula I admixed with a pharmaceutically acceptable diluent, carrier, or excipient.

What is also provided is a method of treating a bacterial infection in a mammal, comprising administering to a mammal in need thereof an effective amount of a compound of one of formula I.

### DETAILED DESCRIPTION OF THE INVENTION

Reference will now be made in detail to presently preferred compositions or embodiments and methods of the invention, which constitute the best modes of practicing the invention presently known to the inventors.

The term “alkyl” as used herein refers to a straight or branched hydrocarbon of from 1 to 6 carbon atoms and includes, for example, methyl, ethyl, n-propyl, isopropyl, n-butyl, sec-butyl, isobutyl, tert-butyl, n-pentyl, n-hexyl, and the like. The alkyl group can also be substituted with one or more of the substituents selected from lower (C₁-C₆)alkoxy, (C₁-C₆)thioalkoxy, halogen, oxo, thio, —OH, —SH, —F, —CF₃, —OCF₃, —NO₂, —CO₂H, —CO₂(C₁-C₆)alkyl, or

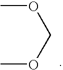

The term “(C₃-C₆)cycloalkyl” means a hydrocarbon ring containing from 3 to 6 carbon atoms, for example, cyclopropyl, cyclobutyl, cyclopentyl, or cyclohexyl. Where possible, the cycloalkyl group may contain double bonds, for example, 3-cyclohexen-1-yl. The cycloalkyl ring may be unsubstituted or substituted by one or more substituents selected from alkyl, alkoxy, thioalkoxy, hydroxy, thiol, halogen, formyl, carboxyl, —CO₂(C₁-C₆)alkyl, —CO(C₁-C₆)alkyl, aryl, heteroaryl, wherein alkyl, aryl, and heteroaryl are as defined herein, or as indicated above for alkyl. Examples of substituted cycloalkyl groups include fluorocyclopropyl.

The term “halo” includes chlorine, fluorine, bromine, and iodine.

The term “aryl” means a cyclic or polycyclic aromatic ring having from 5 to 12 carbon atoms, and being unsubstituted or substituted with one or more of the substituent groups recited above for alkyl groups including, halogen, nitro, cyano —OH, —SH, —F, —CF₃, —OCF₃, 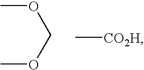
—CO₂(C₁-C₆)alkyl, or —SO₂alkyl. Examples include, but are not limited to phenyl, 2-chlorophenyl, 3-chlorophenyl, 4-chlorophenyl, 2-methylphenyl, 3-methylphenyl, 4-methylphenyl, 2-methoxyphenyl, 3-methoxyphenyl, 4-methoxyphenyl, 2-chloro-3-methylphenyl, 2-chloro-4-methylphenyl, 2-chloro-5-methylphenyl, 3-chloro-2-methylphenyl, 3-chloro-4-methylphenyl, 4-chloro-2-methylphenyl, 4-chloro-3-methylphenyl, 5-chloro-2-methylphenyl, 2,3-dichlorophenyl, 2,5-dichlorophenyl, 3,4-dichlorophenyl, 2,3-dimethylphenyl, 3,4-dimethylphenyl, thienyl, naphthyl, 4-thionaphthyl, tetralinyl, anthracinyl, phenanthrenyl, benzonaphthenyl, fluorenyl, 2-acetamidofluoren-9-yl, and 4′-bromobiphenyl.

The term “heteroaryl” means an aromatic cyclic or polycyclic ring system having from 1 to 4 heteroatoms selected from N, O, and S. Typical heteroaryl groups include 2- or 3-thienyl, 2- or 3-furanyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridinyl, 3-, 4-, or 5-pyridazinyl, 2-pyrazinyl, 2-, 4-, or 5-pyrimidinyl, 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. The heteroaryl groups may be unsubstituted or substituted by 1 to 3 substituents selected from those described above for alkyl, alkenyl, and alkynyl, for example, cyanothienyl and formylpyrrolyl. Preferred aromatic fused heterocyclic rings of from 8 to 10 atoms include but are not limited to 2-, 3-, 4-, 5-, 6-, 7-, or 8-quinolinyl, 1-, 3-, 4-, 5-, 6-, 7-, or 8-isoquinolinyl-, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl. Heteroaryl also includes 2- and 3-aminomethylfuran, 2- and 3-aminomethylthiophene and the like.

The term “heterocyclic” means a monocyclic, fused, bridged, or spiro bicyclic heterocyclic ring systems. Monocyclic heterocyclic rings contain from about 3 to 12 ring atoms, with from 1 to 5 heteroatoms selected from N, O, and S, and preferably from 3 to 7 member atoms, in the ring. Bicyclic heterocyclics contain from about 5 to about 17 ring atoms, preferably from 5 to 12 ring atoms. Bicyclic heterocyclic rings may be fused, spiro, or bridged ring systems. Examples of heterocyclic groups include cyclic ethers (oxiranes) such as ethyleneoxide, tetrahydrofuran, dioxane, and substituted cyclic ethers, wherein the substituents are those described above for the alkyl and cycloalkyl groups. Typical substituted cyclic ethers include propyleneoxide, phenyloxirane (styrene oxide), cis-2-butene-oxide (2,3-dimethyloxirane), 3-chlorotetrahydrofuran, 2,6-dimethyl-1,4-dioxane, and the like. Heterocycles containing nitrogen are groups such as pyrrolidine, piperidine, piperazine, tetrahydrotriazine, tetrahydropyrazole, and substituted groups such as 3-aminopyrrolidine, 4-methylpiperazin-1-yl, and the like. Typical sulfur containing heterocycles include tetrahydrothiophene, dihydro-1,3-dithiol-2-yl, and hexahydrothiophen-4-yl and substituted groups such as aminomethyl thiophene. Other commonly employed heterocycles include dihydro-oxathiol-4-yl, dihydro-1H-isoindole, tetrahydro-oxazolyl, tetrahydro-oxadiazolyl, tetrahydrodioxazolyl, tetrahydrooxathiazolyl, hexahydrotriazinyl, tetrahydro-oxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothiophene.

When a bond is represented by a symbol such as 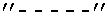
this is meant to represent that the bond may be absent or present provided that the resultant compound is stable and of satisfactory valency.

When a bond is represented by a line such as 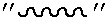
this is meant to represent that the bond is the point of attachment between two molecular subunits.

The term “patient” means all mammals, including humans. Other examples of patients include cows, dogs, cats, goats, sheep, pigs, and rabbits.

A “therapeutically effective amount” is an amount of a compound of the present invention that, when administered to a patient, provides the desired effect; i.e., lessening in the severity of the symptoms associated with a bacterial infection.

It will be appreciated by those skilled in the art that compounds of the invention having one or more chiral centers may exist in and be isolated in optically active and racemic forms. Some compounds may exhibit polymorphism. It is to be understood that the present invention encompasses any racemic, optically-active, polymorphic, geometric, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity or cytotoxicity using the standard tests described herein, or using other similar tests which are well known in the art.

Certain compounds of Formula I are also useful as intermediates for preparing other compounds of Formula I. Thus, a compound wherein R₂ is NR₂, can be metabolized to form another compound of the invention wherein R₂ is H. This conversion can occur under physiological conditions. To that end, both the non-metabolized compound of the invention and the metabolized compound of the invention—that is, the compound wherein R₂ is NR₂ and the compound wherein R₂ is H—can have antibacterial activity.

Some of the compounds of Formula I are capable of further forming pharmaceutically acceptable acid-addition and/or base salts. All of these forms are within the scope of the present invention. Thus, pharmaceutically acceptable acid addition salts of the compounds of Formula I include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, acetate, trifluoroacetate, propionate, caprylate, isobutyrate, oxalate, malonate, succinates suberate, sebacate, fumarate, maleate, mandelate, benzoate, chlorobenzoate, methylbenzoate, dinitrobenzoate, phthalate, benzensoulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al., “Pharmaceutical Salts,” Journal of Pharmaceutical Science, 1977;66:1-19).

The acid addition salt of said basic compounds are prepared by contacting the free base form with a sufficient amount of the desired acid to produce the salt in the conventional manner.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge S. M., supra., 1977).

The base addition salts of said acidic compounds are prepared by contacting the free acid form with a sufficient amount of the desired base to produce the salt in the conventional manner.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

A “prodrug” is a derivative of a drug molecule that requires a chemical or an enzymatic biotransformation in order to release the active parent drug in the body.

Specific and preferred values for the compounds of the present invention are listed below for radicals, substituents, and ranges are for illustration purposes only, and they do not exclude other defined values or other values within defined ranges for the radicals and substituents.

In one embodiment of the invention compound
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is H,
   NH₂,
   NH(C₁-C₆)alkyl,
   NH(C₃-C₆)cycloalkyl,
   NH-heteroaryl,
   NHSO₂—(C₁-C₆)alkyl,
   NHSO₂-aryl,
   NHSO₂-heteroaryl,
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
Rₐ is H,
   aryl,
   (C₁-C₆)alkyl,
   halo(C₁-C₆)alkyl,
   (C₃-C₆)cycloalkyl, 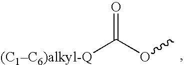
   wherein 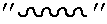
   indicates the point of attachment and Q is O or is absent, 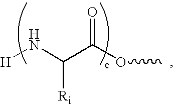
   wherein 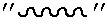
   indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 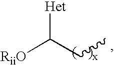
   wherein 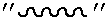
   indicates the point of attachment, het is a 5- or 6-membered heterocyclo or heteroaryl group, and x is an integer of from 0 to 10; 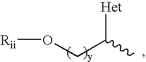
   wherein 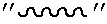
   indicates the point of attachment, het is as defined above, and y is an integer of from 1 to 10;
      wherein Rᵢᵢ is H,
         (C₁-C₆)alkyl,
         PO(OH)₂,
         PO((C₁-C₆)alkyl)₂, 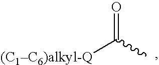
         as defined above, or 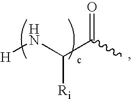
         as defined above;
      provided that 3 or fewer of R_{c} R_{d}, Rₑ, and R_{f} are H; or
R_{b} is OH,
   OPO(OH)₂,
   OPO(O(C₁-C₆)alkyl)₂, 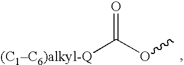
   wherein 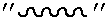
   indicates the point of attachment and Q is O or is absent, 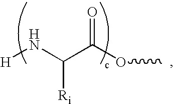
   wherein 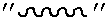
   indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 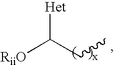
   wherein 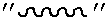
   indicates the point of attachment, het is a 5- or 6-membered heterocyclo or heteroaryl group, and x is an integer of from 0 to 10; 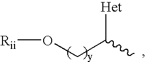
   wherein 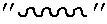
   indicates the point of attachment, het is as defined above, and y is an integer of from 1 to 10;
      wherein Rᵢᵢ s H,
         (C₁-C₆)alkyl,
         PO(OH)₂,
         PO((C₁-C₆)alkyl)₂, 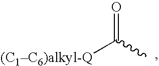
         as defined above, or 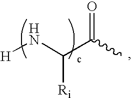
         as defined above.

In another embodiment of the invention compound,
R₁ is (C₁-C₆)alkyl,
   halo(C₁-C₆)alkyl,
   (C₃-C₆)cycloalkyl,
   halo(C₃-C₆)cycloalkyl
   aryl, and heteroaryl;
R₂ is H,
   NH₂,
   NH(C₁-C₆)alkyl,
   NH(C₃-C₆)cycloalkyl,
   NH-heteroaryl,
   NHSO₂—(C₁-C₆)alkyl,
   NHSO₂-aryl,
   NHSO₂-heteroaryl;
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
Rₐ is H,
   aryl,
   (C₁-C₆)alkyl,
   halo(C₁-C₆)alkyl,
   (C₃-C₆)cycloalkyl, 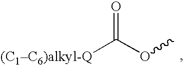
   wherein 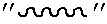
   indicates the point of attachment and Q is O or is absent, 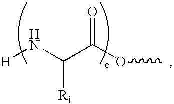
   wherein 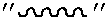
   indicates the point of attachment, Rᵢ is H or (C₁-C₆)alkyl, and c is an integer having a value of from 1 to 10,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 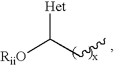
   wherein 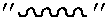
   indicates the point of attachment, het is a 5- or 6-membered heterocyclo or heteroaryl group, and x is an integer of from 0 to 10; 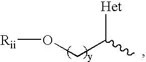
   wherein 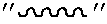
   indicates the point of attachment, het is as defined above, and y is an integer of from 1 to 10;
      wherein Rᵢᵢ is H, (C₁-C₆)alkyl,
      PO(OH)₂,
      PO((C₁-C₆)alkyl)₂, 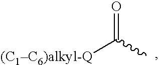
      as defined above, or 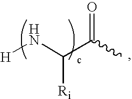
      as defined above;
      provided that 3 or fewer of R_{d} Rₑ, and R_{f} are H; or
R_{b} is OH,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 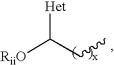
   wherein 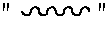
   indicates the point of attachment, het is a 5- or 6-membered heterocyclo or heteroaryl group, and x is an integer of from 0 to 10; 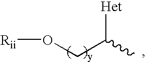
   wherein 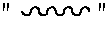
   indicates the point of attachment, het is as defined above, and y is an integer of from 1 to 10;
      wherein Rᵢᵢ is H or (C₁-C₆)alkyl.

In another embodiment of the invention compound,
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is H,
   NH₂,
   NH(C₁-C₆)alkyl,
   NH(C₃-C₆)cycloalkyl,
   NH-heteroaryl,
   NHSO₂—(C₁-C₆)alkyl,
   NHSO₂-aryl,
   NHSO₂-heteroaryl,
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
Rₐ is H,
   aryl,
   (C₁-C₆)alkyl,
   halo(C₁-C₆)alkyl,
   (C₃-C₆)cycloalkyl, 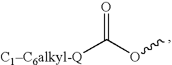
   wherein 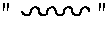
   indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 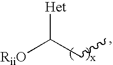
   wherein 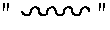
   indicates the point of attachment, het is a 5- or 6-membered heterocyclo or heteroaryl group, and x is an integer of from 0 to 10; 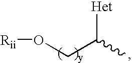
   wherein 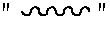
   indicates the point of attachment, het is as defined above, and y is an integer of from 1 to 10;
      wherein Rᵢᵢ is H,
         (C₁-C₆)alkyl,
         PO(OH)₂,
         PO(O(C₁-C₆)alkyl)₂, 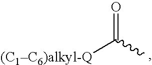
         as defined above, or 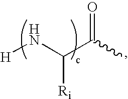
         as defined above;
provided that 3 or fewer of R_{c} R_{d}, Rₑ, and R_{f} are H; or
R_{b} is OH,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 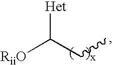
   wherein 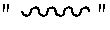
   indicates the point of attachment, het is a 5- or 6-membered heterocyclo or heteroaryl group, and x is an integer of from 0 to 10; 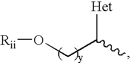
   wherein 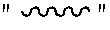
   indicates the point of attachment, het is as defined above, and y is an integer of from 1 to 10;
      wherein Rᵢᵢ is H or (C₁-C₆)alkyl.

In another embodiment of the invention compound,
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is H,
   NH₂,
   NH(C₁-C₆)alkyl,
   NH(C₃-C₆)cycloalkyl,
   NH-heteroaryl,
   NHSO₂—(C₁-C₆)alkyl,
   NHSO₂-aryl, NHSO₂-heteroaryl,
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
Rₐ is H, aryl, (C₁-C₆)alkyl, halo(C₁-C₆)alkyl, (C₃-C₆)cycloalkyl, 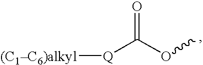
   wherein 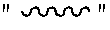
   indicates the point of attachment and Q is O or is absent,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 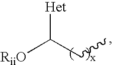
   wherein 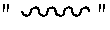
   indicates the point of attachment, het is a 5- or 6-membered heterocyclo or heteroaryl group, and x is an integer of from 0 to 10; 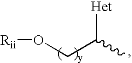
   wherein 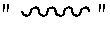
   indicates the point of attachment, het is as defined above, and y is an integer of from 1 to 10;
      wherein Rᵢᵢ is H,
         (C₁-C₆)alkyl, 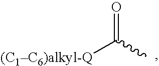
         as defined above, or 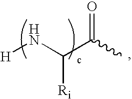
         as defined above;
      provided that 3 or fewer of R_{c} R_{d}, Rₑ, and R_{f} are H; or
R_{b} is OH,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—,
   RᵢᵢO(C₃-C₆)cycloalkyl-O—, 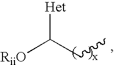
   wherein 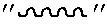
   indicates the point of attachment, het is a 5- or 6-membered heterocyclo or heteroaryl group, and x is an integer of from 0 to 10; 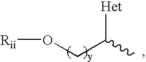
   wherein 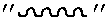
   indicates the point of attachment, het is as defined above, and y is an integer of from 1 to 10;
      wherein Rᵢᵢ is H or (C₁-C₆)alkyl.

In another embodiment of the invention compound,
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is H,
   NH₂,
   NH(C₁-C₆)alkyl,
   NH(C₃-C₆)cycloalkyl,
   NH-heteroaryl,
   NHSO₂—(C₁-C₆)alkyl,
   NHSO₂-aryl,
   NHSO₂-heteroaryl,
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
Rₐ is H,
   aryl,
   (C₁-C₆)alkyl,
   halo(C₁-C₆)alkyl,
   (C₃-C₆)cycloalkyl,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—,
   RᵢᵢO(C₁-C₆)haloalkyl-O—, or
   RᵢᵢO(C₃-C₆)cycloalkyl-O—, wherein Rᵢᵢ is H or (C₁-C₆)alkyl; and
R_{b} is OH,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—, or
   RᵢᵢO(C₁-C₆)haloalkyl-O—, wherein Rᵢᵢ is H or (C₁-C₆)alkyl.

In another embodiment of the invention compound,
R₁ is (C₁-C₆)alkyl,
   halo(C₁-C₆)alkyl,
   (C₃-C₆)cycloalkyl,
   halo(C₃-C₆)cycloalkyl
   aryl, and
   heteroaryl;
R₂ is H,
   NH₂,
   NH(C₁-C₆)alkyl,
   NH(C₃-C₆)cycloalkyl,
   NH-heteroaryl,
   NHSO₂—(C₁-C₆)alkyl,
   NHSO₂-aryl,
   NHSO₂-heteroaryl,
R₃, R₄, and R₅ are each independently H,
   halo,
   NH₂,
   (C₁-C₆)alkyl,
   halo(C₁-C₆)alkyl,
   (C₁-C₆)alkoxy, or
   halo(C₁-C₆)alkoxy;
Rₐ is H,
   aryl,
   (C₁-C₆)alkyl,
   halo(C₁-C₆)alkyl,
   (C₃-C₆)cycloalkyl,
   RᵢᵢO(C₁-C₆)alkyl,
   RᵢᵢO(C₁-C₆)haloalkyl,
   RᵢᵢO(C₃-C₆)cycloalkyl, or
   RᵢᵢO(C₃-C₆)cycloalkyl-O—, wherein Rᵢᵢ is H or (C₁-C₆)alkyl; and
R_{b} is OH,
   RᵢᵢO(C₁-C₆)alkyl,
   RᵢᵢO(C₁-C₆)haloalkyl,
   RᵢᵢO(C₃-C₆)cycloalkyl,
   RᵢᵢO(C₁-C₆)alkyl-O—, or
   RᵢᵢO(C₁-C₆)haloalkyl-O—, wherein Rᵢᵢ is H or (C₁-C₆)alkyl.

In another embodiment of the invention compound,
R₁ is (C₁-C₆)cycloalkyl and halo(C₁-C₆)cycloalkyl,
   aryl, or
   heteroaryl;
R₂ is H or NH₂;
R₃ is H or NH₂;
R₄ is H or halo; and
R₅ is halo,
   methyl,
   trifluoromethyl,
   methoxy,
   fluoromethoxy,
   difluoromethoxy, or
   trifluoromethoxy.

In another embodiment of the invention compound,
R₁ is cyclopropyl,
   fluorocyclopropyl, 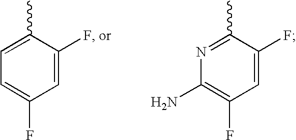
R₂ is H or NH₂;
R₃ is H or NH₂;
R₄ is H or F; and
R₅ is halo,
   methyl,
   trifluoromethyl, or
   methoxy.

In another embodiment of a compound of formula I, specific values for R₁, R₂, R₃, R₄, and R₅ are as provided in the following structures wherein R₄ is H or F and A is

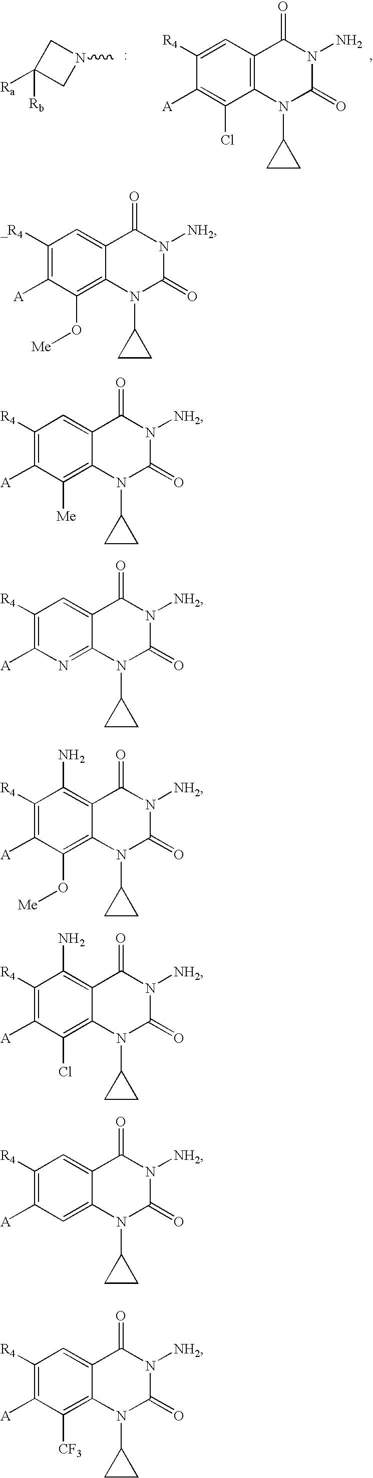

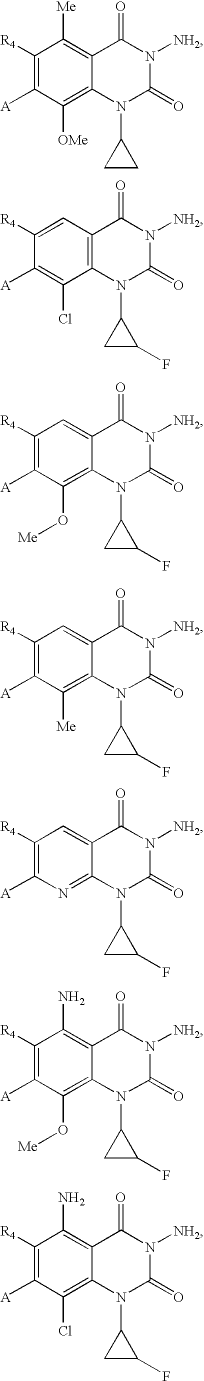

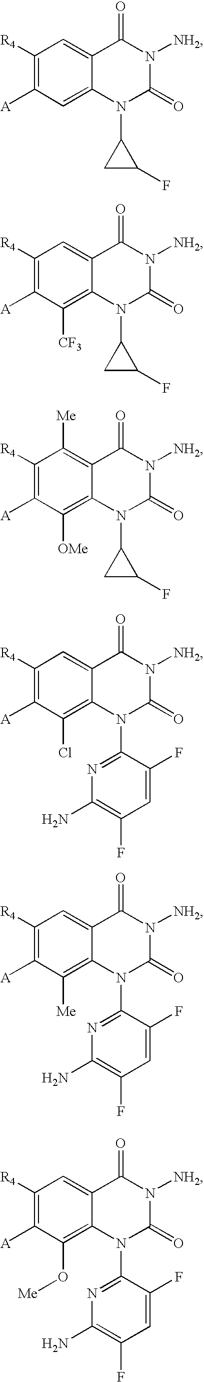

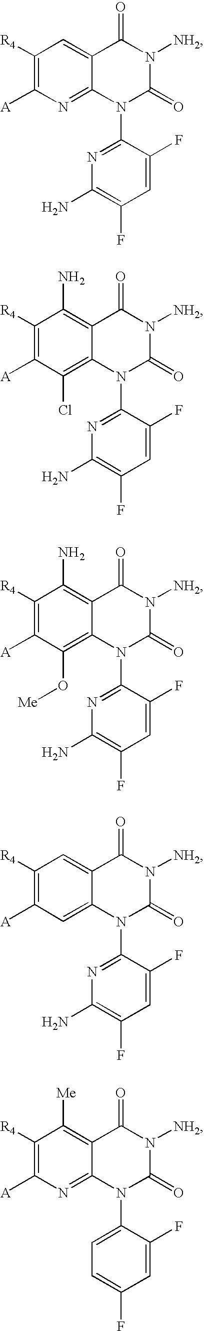

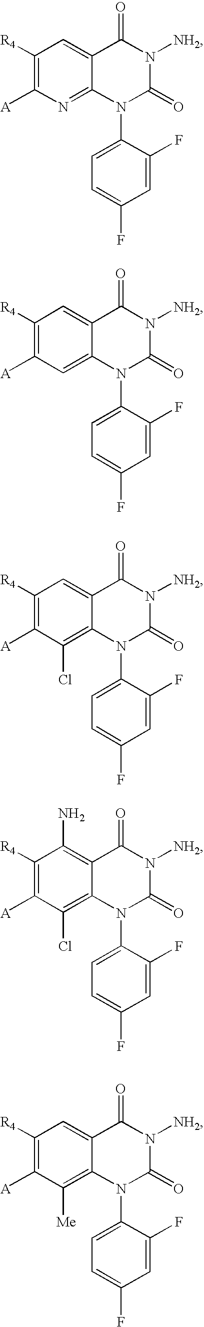

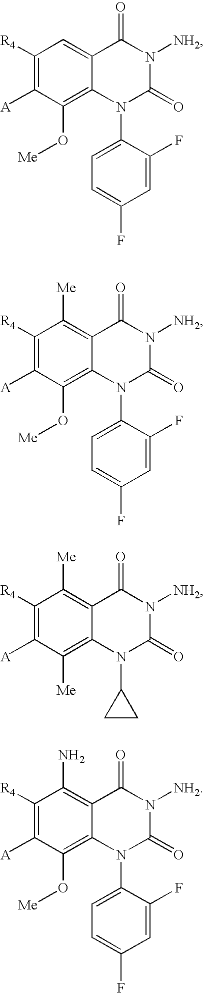

In another embodiment of a compound of formula I, specific values for R₁, R₂, R₃, R₄, and R₅ are as provided in the following structures wherein R₄ is H or F and A is

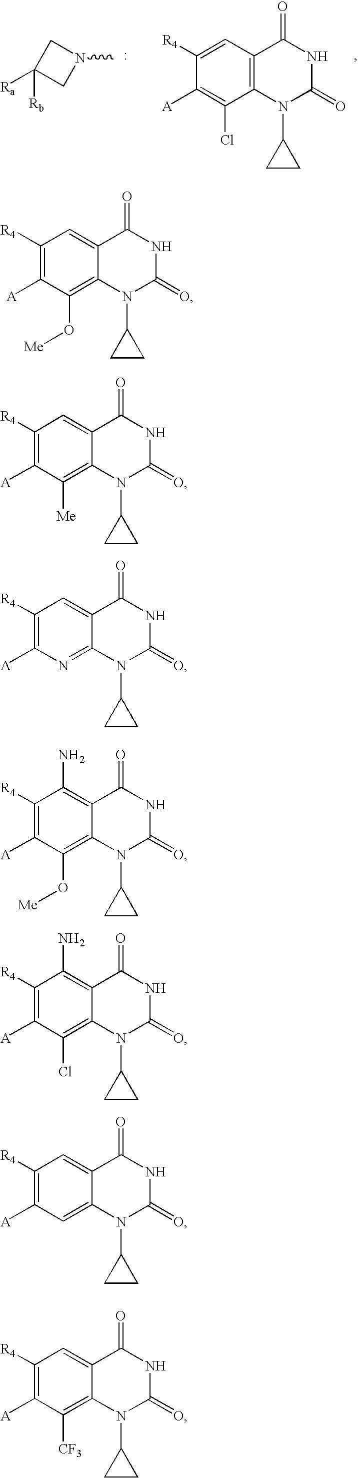

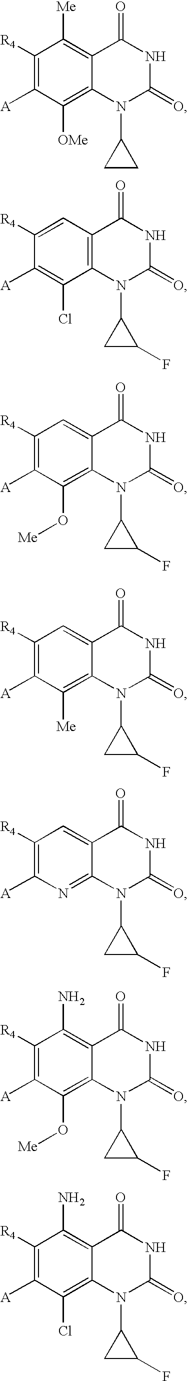

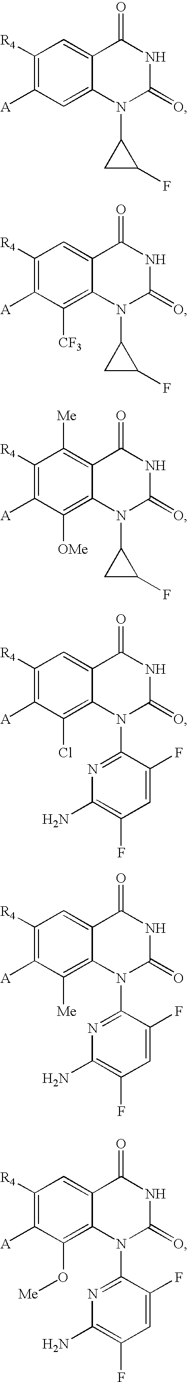

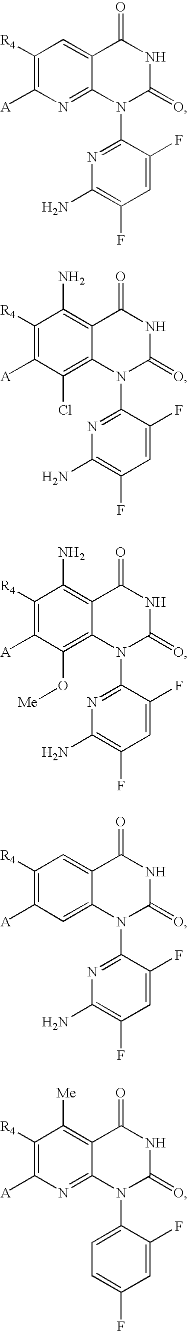

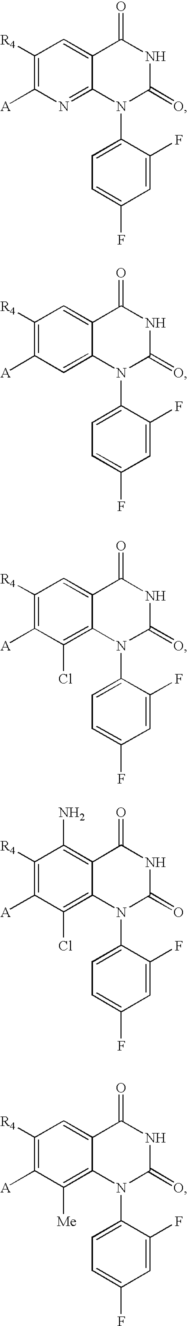

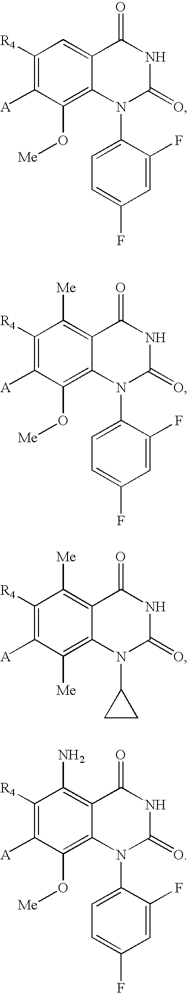

In another embodiment of the invention compound,

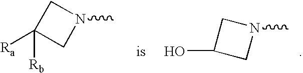

In other embodiments,

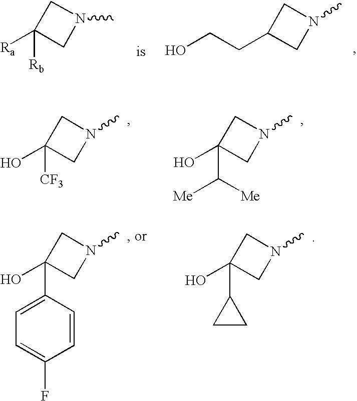

### Preparation of Invention Compounds

Strategies for the preparation of invention compounds are depicted in Scheme I, and more specifically in subsequent schemes.

As is readily apparent from this disclosure, compounds of the present invention are characterized by a quinazolinedione core, covalently bound to an hydroxylated azetidinyl C-7 sidechain. As retrosynthetically depicted in Scheme I, the invention compounds can be prepared via coupling of a suitably C-7 substituted quinazolinedione core precursor, wherein X is halo, triflate, or a similar reactive group known to the skilled artisan, and 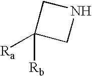
is an appropriately substituted azetidine.

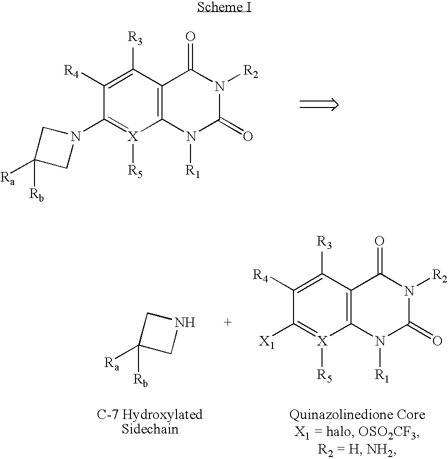

Reflecting the synthetic strategy summarized in Scheme I, the following section describing the preparation of the invention compounds has several parts. The first part describes the synthesis of the requisite quinazolinedione core precursors. The second part describes the synthesis of the requisite C-7 sidechain precursors. The final part describes the coupling of the C-7 sidechain and quinazolinedione core precursors to provide the invention compounds, and details any further chemical elaboration of invention compounds to produce other invention compounds.

A. Synthesis of Quinazolinedione Core Precurors

The quinazolinedione core precursors that are used to prepare the invention compounds can be prepared as described in WO/02 102793, priority date Jun. 19, 2001 and WO/01 53273, priority date Oct. 18, 2000, and references cited therein.

B. Synthesis of Hydroxylated C-7 Sidechain Precurors

The requisite hydroxylated azetidinol sidechains used to prepare the invention compounds are readily prepared as indicated in Scheme B1. Thus, azetidinol sidechains can be prepared via Grignard addition of a substituted or unsubstituted alkyl, aryl, or heteroaryl Grignard reagents to the corresponding ketone A to provide B. Deprotection of B provides the requisite azetidinol C. See, e.g., Rosenberg, S. H.; Spina, K. P.; Condon, S. L.; Polakowski, J.; Yao, Z.; Kovar, P.; Stein, H. H.; Cohen, J.; Barlow, J. L.; Klinghofer, V.; Egan, D. A.; Tricarico, K. A.; Perun, T. J.; Baker, W. R.; Kleinert, H. D. J. Med. Chem. 1993, 36, 460-467.

Sidechains bearing an hydroxylated alkyl substituent can be prepared via methylenation of the ketone moiety in A, for instance, using Wittig technology to provide compound D. Reduction of sidechain functional groups such as esters as depicted in Scheme B-1 is readily effected using LAH or the like, followed by hydrogenation, provides compound E. Deprotection of E provides the requisite azetidinol F. 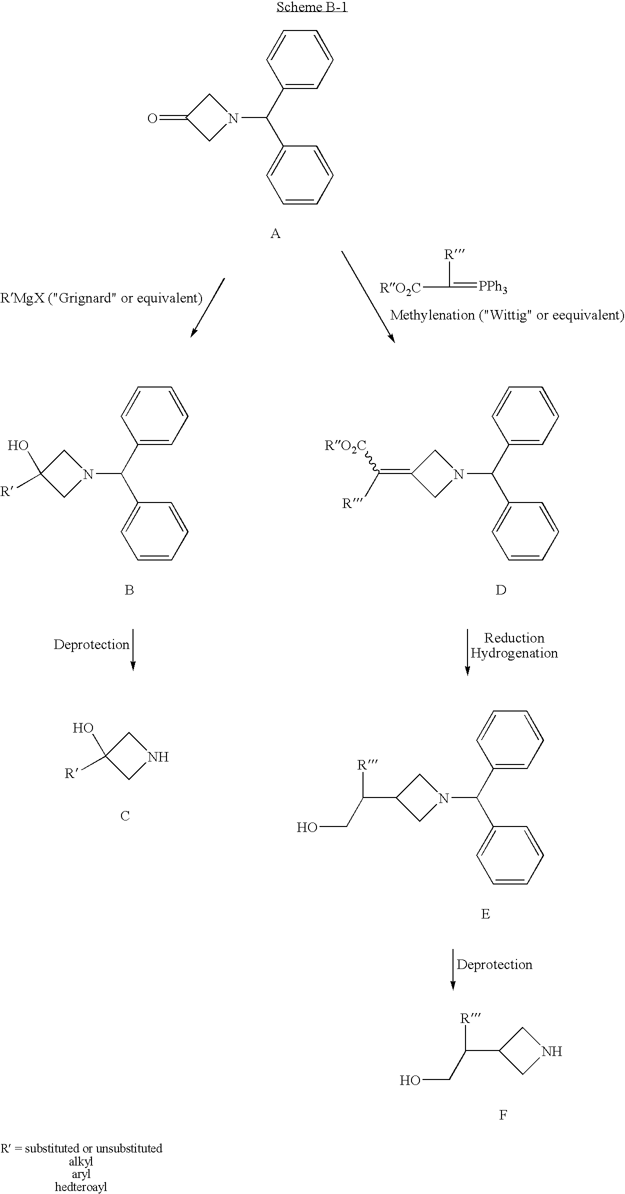
C. Coupling of Hydroxylated C-7 Sidechain and Quinazolinedione Core Precursors to Provide Invention Compounds

Coupling of the sidechain precursor to the quinazolinedione core precursor to provide the compounds of the present invention occurs as described in WO/02 102793, priority date Jun. 19, 2001 and WO/01 53273, priority date Oct. 18, 2000, and references cited therein.

### Pharmaceutical Formulations

The present invention also provides pharmaceutical compositions which comprise a bioactive invention compound or a pharmaceutically acceptable salt thereof and optionally a pharmaceutically acceptable carrier. The compositions include those in a form adapted for oral, topical or parenteral use and can be used for the treatment of bacterial infection in mammals including humans.

Compounds of the invention can be formulated for administration in any convenient way for use in human or veterinary medicine, by analogy with other bioactive agents such as antibiotics. Such methods are known in the art and are not described in detail herein.

The composition can be formulated for administration by any route known in the art, such as subdermal, by-inhalation, oral, topical or parenteral. The compositions may be in any form known in the art, including but not limited to tablets, capsules, powders, granules, lozenges, creams or liquid preparations, such as oral or sterile parenteral solutions or suspensions.

The topical formulations of the present invention can be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present, for example, from about 1% up to about 98% of the formulation. For example, they may form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods will known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavoring or coloring agents.

For parenteral administration, fluid unit dosage forms are prepared utilizing the compound and a sterile vehicle, water being preferred. The compound, depending on the vehicle and concentration used, can be either suspended or dissolved in the vehicle or other suitable solvent. In preparing solutions, the compound can be dissolved in water for injection and filter sterilized before filling into a suitable vial or ampoule and sealing. Advantageously, agents such as a local anesthetic preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

The compositions may contain, for example, from about 0.1% by weight, e.g., from about 10-60% by weight, of the active material, depending on the method of administration. Where the compositions comprise dosage units, each unit will contain, for example, from about 50-500 mg of the active ingredient. The dosage as employed for adult human treatment will range, for example, from about 100 to 3000 mg per day, for instance 1500 mg per day depending on the route and frequency of administration. Such a dosage corresponds to about 1.5 to 50 mg/kg per day. Suitably the dosage is, for example, from about 5 to 20 mg/kg per day.

### Biological Activity

The invention compounds can be screened to identify bioactive molecules with different biological activities using methods available in the art. The bioactive molecules, for example, can possess activity against a cellular target, including but not limited to enzymes and receptors, or a microorganism. A target cellular ligand or microorganism is one that is known or believed to be of importance in the etiology or progression of a disease. Examples of disease states for which compounds can be screened for biological activity include, but are not limited to, inflammation, infection, hypertension, central nervous system disorders, and cardiovascular disorders.

In one embodiment, the invention provides methods of treating or preventing a bacterial infection in a subject, such as a human or other animal subject, comprising administering an effective amount of an invention compound as disclosed herein to the subject. In one embodiment, the compound is administered in a pharmaceutically acceptable form optionally in a pharmaceutically acceptable carrier. As used herein, an “infectious disorder” is any disorder characterized by the presence of a microbial infection, such as bacterial infections. Such infectious disorders include, for example central nervous system infections, external ear infections, infections of the middle ear, such as acute otitis media, infections of the cranial sinuses, eye infections, infections of the oral cavity, such as infections of the teeth, gums and mucosa, upper respiratory tract infections, lower respiratory tract infections, genitourinary infections, gastrointestinal infections, gynecological infections, septicemia, bone and joint infections, skin and skin structure infections, bacterial endocarditis, burns, antibacterial prophylaxis of surgery, and antibacterial prophylaxis in immunosuppressed patients, such as patients receiving cancer chemotherapy, or organ transplant patients. The compounds and compositions comprising the compounds can be administered by routes such as topically, locally or systemically. Systemic application includes any method of introducing the compound into the tissues of the body, e.g., intrathecal, epidural, intramuscular, transdermal, intravenous, intraperitoneal, subcutaneous, sublingual, rectal, and oral administration. The specific dosage of antimicrobial to be administered, as well as the duration of treatment, may be adjusted as needed.

The compounds of the invention may be used for the treatment or prevention of infectious disorders caused by a variety of bacterial organisms. Examples include Gram positive and Gram negative aerobic and anaerobic bacteria, including Staphylococci, for example S. aureus; Enterococci, for example E. faecalis; Streptococci, for example S. pneumoniae; Haemophilus, for example H. influenza; Moraxella, for example M. catarrhalis; and Escherichia, for example E. coli. Other examples include Mycobacteria, for example M. tuberculosis; intercellular microbes, for example Chlamydia and Rickettsiae; and Mycoplasma, for example M. pneumoniae.

The ability of a compound of the invention to inhibit bacterial growth, demonstrate in vivo activity, and enhanced pharmacokinetics are demonstrated using pharmacological models that are well known to the art, for example, using models such as the tests described below.

Test A—Antibacterial Assays

The compounds of the present invention were tested against an assortment of Gram-negative and Gram-positive organisms using standard microtitration techniques (Cohen et. al., Antimicrob., 1985;28:766; Heifetz, et. al., Antimicrob., 1974;6:124). The results of the evaluation are shown in Tables 1A and B.

TABLE 1A
Minimum Inhibitory Concentrations μg/mL Gram Negative Bacteria

| | H. influenzae | M. catarrhalis | E. coli |
|---|---|---|---|
| Compound | HI-3542 | BC-3531 | MC4100 |
| 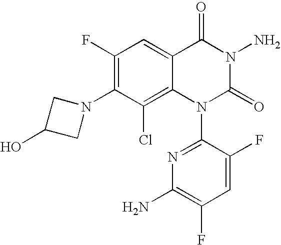 | 1 | 4 | 64 |
| 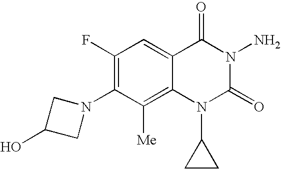 | 32 | 1 | 1 |
| 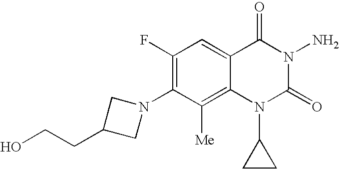 | 4 | 4 | 32 |
| 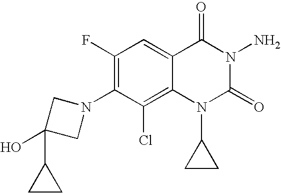 | 0.03 | 0.03 | 0.002 |
| 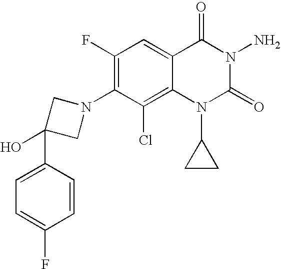 | 32 | 16 | 64 |
| 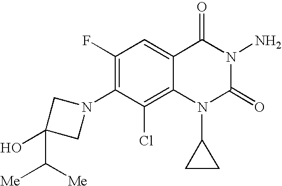 | 32 | 32 | 64 |

TABLE 1B
Minimum Inhibitory Concentrations μg/mL Gram Positive Bacteria

| | E. faecalis | S. aureus | S pyogenes |
|---|---|---|---|
| Compound | MGH-2 | UC-76 | C203 |
| 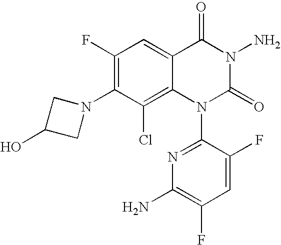 | 16 | 4 | 16 |
| 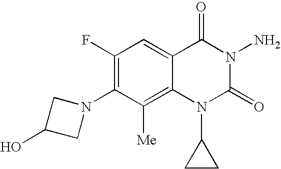 | 32 | 1 | 16 |
| 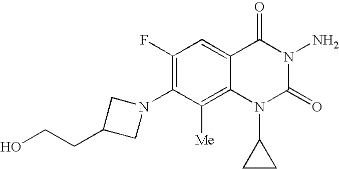 | 16 | 4 | 16 |
| 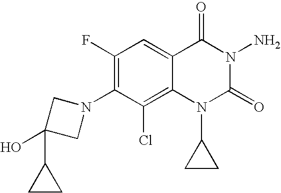 | 0.5 | 0.03 | 0.5 |
| 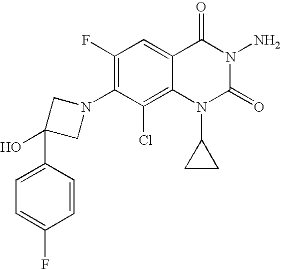 | 16 | 4 | 8 |
| 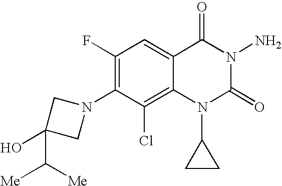 | 32 | 16 | 64 |

### EXAMPLES

The following examples are provided to illustrate but not limit the claimed invention.

A. Sidechain Preparation

### Example A-1

### 1-Benzhydryl-3-cyclopropyl-azetidin-3-ol (1)

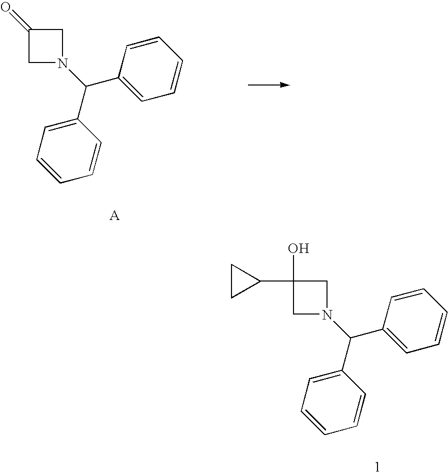

To 0.5 M cyclopropylmagnesium bromide in tetrahydrofuran (THF) (40 mL, 20.0 mmol) at −78° C. was added 1-benzhydryl-azetidin-3-one (A) (1.70 g, 7.16 mmol), dropwise as a solution in 10 mL tetrahydrofuran. After 30 minutes the reaction was poured onto saturated aqueous sodium bicarbonate and extracted with diethylether (3 times). The combined organic layers were dried over sodium sulfate and concentrated in vacuo to give an oil that was purified by silica gel chromatography (gradient 2%-15% isopropanol/hexanes) to give 1.649 g (82%) of the title compound. MS(APCI+): 280.2 (m+1/z).

### Example A-2

### 1-Benzhydryl-3-isopropyl-azetidin-3-ol (2)

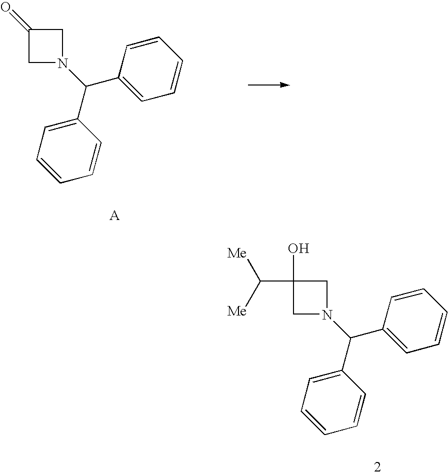

To 2.0 M isopropylmagnesium bromide in tetrahydrofuran (13.0 mL, 26.0 mmol) cooled in an acetone/water-ice bath was added 1-benzhydryl-azetidin-3-one (2.00 g, 8.43 mmol), dropwise, as a 7 mL solution in tetrahydrofuran. After 1 hour the reaction was poured onto 10% aqueous sodium bicarbonate and extracted with diethylether (3 times). The combined organic layers were dried over sodium sulfate and concentrated in vacuo to give a yellow oil. Purification by silica gel chromatography (gradient 2% to 10% isopropanol/dichloromethane) gave 1.60 g (67%) of the title compound as a yellow oil. MS(APCI+): 282.2 (m+1/z).

### Example A-3

### 1-Benzhydryl-3-(4-fluoro-phenyl)-azetidin-3-ol (3)

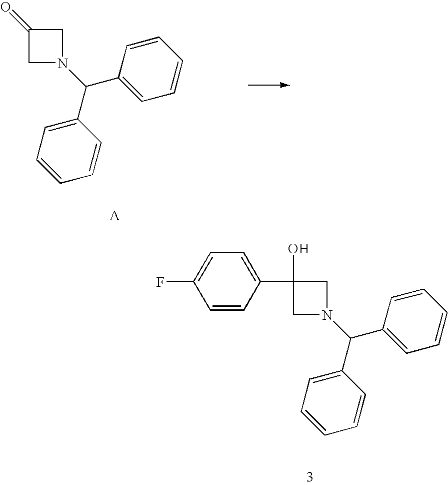

To 1.0 M 4-fluorophenylmagnesium bromide in tetrahydrofuran (30.0 mL, 30.0 mmol) cooled in an acetone/water-ice bath was dropwise added 1-benzhydryl-azetidin-3-one (A) (2.00 g, 8.43 mmol) as a 7 mL solution in tetrahydrofuran. After 1 hour the reaction was poured onto 10% aqueous sodium bicarbonate and extracted with diethylether (3 times). The combined organic layers were dried over sodium sulfate and evaporated in vacuo to give a pale yellow oil. Purification by silica gel chromatography (2% to 10% isopropanol in dichloromethane) gave 2.119 g (75%) of the title compound. MS(APCI+): 334.10 (m+1/z).

### Example A-4

### 1-Benzhydryl-3-trifluoromethyl-azetidin-3-ol (4)

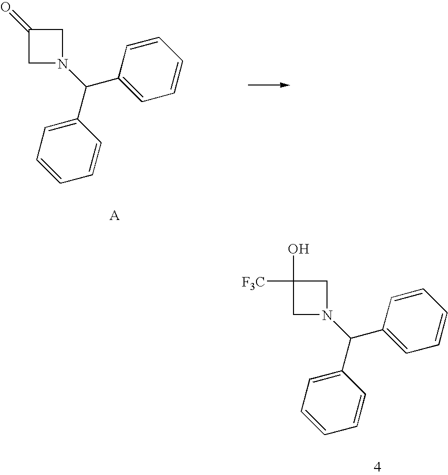

To a solution of 1-benzhydryl-azetidin-3-one (A) (2.00 g, 8.43 mmol) in 7 mL tetrahydrofuran at 23° C. was added trifluoromethyltrimethylsilane (1.80 g, 12.6 mmol) followed by cesium fluoride (1.95 g, 12.8 mmol). After 15 minutes, the reaction was quenched by the addition of 7 mL of saturated aqueous ammonium chloride and 1.00 g of tetrabutylammonium fluoride hydrate. The resulting biphasic mixture was stirred vigorously for 1 day then extracted with diethyl ether (3 times). The combined organic layers were dried over sodium sulfate and evaporation in vacuo gave an orange oil that was purified by silica gel chromatography (gradient 2% to 10% isopropanol in dichloromethane) to give 1.98 g (76%) of the title compound. MS(APCI+): 308.1 (m+1/z); 349.1 (m+41/z) (acetonitrile).

### Example A-5

### 2-(1-Benzhydryl-azetidin-3-yl)-ethanol (6)

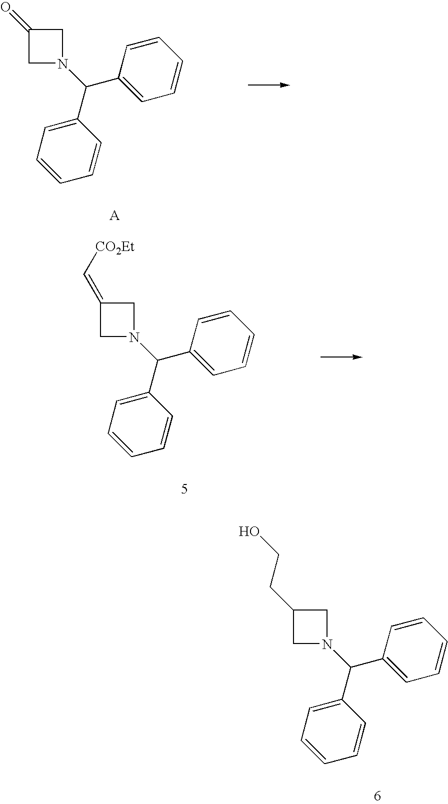

To a solution of 1-benzhydryl-azetidin-3-one (A) (6.00 g, 25.3 mmol) in 46 mL tetrahydrofuran at 23° C. was added (carbethoxymethylene)triphenyl phosphorane (9.45 g, 27.1 mmol) as a single portion. After 3 days the reaction was evaporated in vacuo to a viscous oil. Trituration with 20% ethyl acetate in hexanes resulted in a liquid/solid biphase from which the organic liquid was decanted and evaporated in vacuo to give a yellow oil. Further purification by silica gel chromatography (gradient 1% to 15% ethyl acetate in hexanes) gave 5.75 g (74%) of (1-benzhydryl-azetidin-3-ylidene)-acetic acid ethyl ester (5) as a slightly yellow oil. MS(APCI+): 308.1 (m+1/z). To a solution of 5 (3.18 g, 10.3 mmol) in 20 mL tetrahydrofuran at −78° C. was added dropwise 1.0 M lithium aluminum hydride (LAH) in diethylether (20 mL, 80 mmol hydride). The reaction was stirred for 20 minutes at −78° C. then allowed to warm to 23° C. After 4 hours, the reaction was treated sequentially with 0.75 mL water, 0.75 mL 1.0 N sodium hydroxide 2.25 mL of water, then filtered. Evaporation of the filtrate gave 2.75 g (99%) of the title compound. MS(APCI+): 268.1 (m+1/z).

### Example A-6

### General Procedure for N-Deprotection

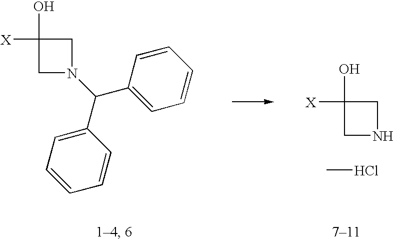

General Procedure for N-deprotection: To a solution of the benzhydrylazetidine (10 mmol) in 50 mL methanol was added concentrated hydrochloric acid (10 mmol) followed by an equivalent weight of 20% palladium(II) hydroxide on carbon. The mixture was shaken in a Parr shaker charged with hydrogen gas (approximately 50 psi) until complete consumption of the benzhydrylazetidine was indicated by mass spectrometry. The reaction was then filtered. The filtrate was evaporated in vacuo to give a colorless biphasic mixture that was washed with hexane (5 times) to remove the diphenylmethane by-product. The remaining azetidine hydrochloride salt was typically used without further purification or could be purified by precipitation from ethyl acetate to obtain a powder form.

### Example A-7

### 3-Cyclopropyl-azetidin-3-ol Hydrochloride (7)

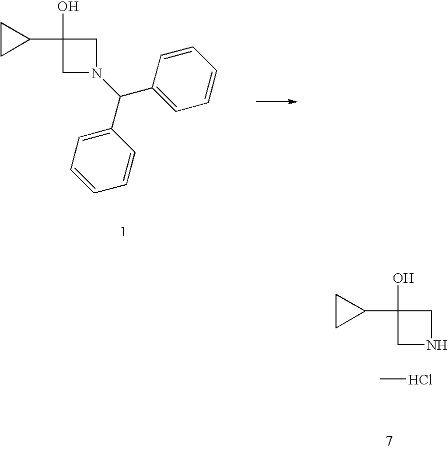

The title compound was obtained in 76% yield as a colorless solid. MS(APCI+): 114.0 (m+1/z).

### Example A-8

### 3-Isopropyl-azetidin-3-ol Hydrochloride (8)

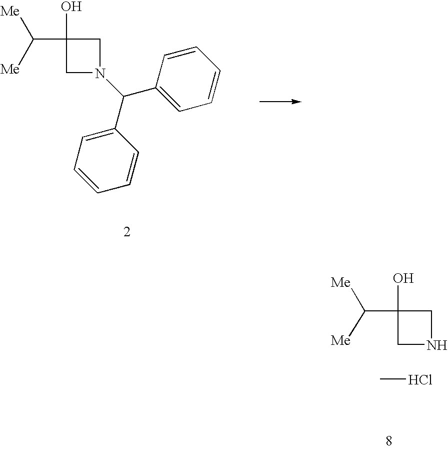

The title compound was obtained using General Procedure 7 in 95% yield as a colorless oil. MS(APCI+): 116.0 (m+1/z).

### Example A-9

### 3-(4-Fluoro-phenyl)-azetidin-3-ol Hydrochloride (9)

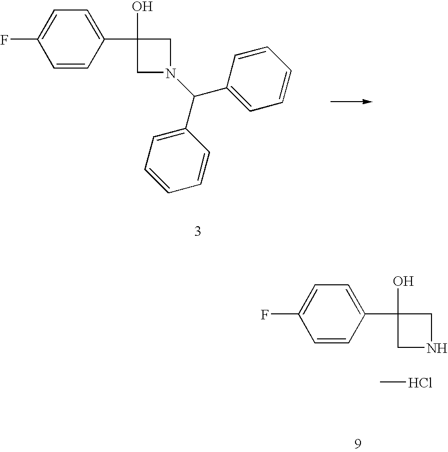

The title compound was obtained using General Procedure 7 in 69% yield as a colorless solid. MS(APCI+): 168.2 (m+1/z).

### Example A-10

### 3-Trifluoromethyl-azetidin-3-ol Hydrochloride (10)

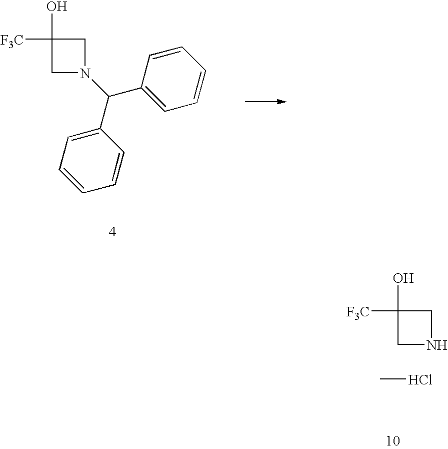

The title compound was obtained using General Procedure 7 in 36% yield as a colorless powder. MS(APCI+): 142.0 (m+1/z); 183.0 (m+41/z) (acetonitrile).

### Example A-11

### 2-Azetidin-3-yl-ethanol hydrochloride (11)

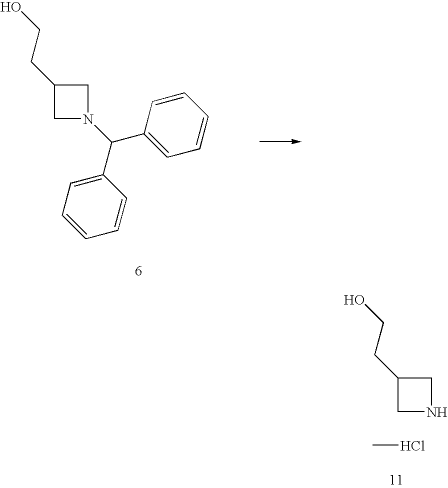

The title compound was obtained using General Procedure 7 in 89% yield as a colorless oil. MS(APCI+): 102.0 (m+1/z).

B. Coupling Reactions

### Example B-1

### General Procedure for Coupling Azetidine Sidechain and Quinazolinedione

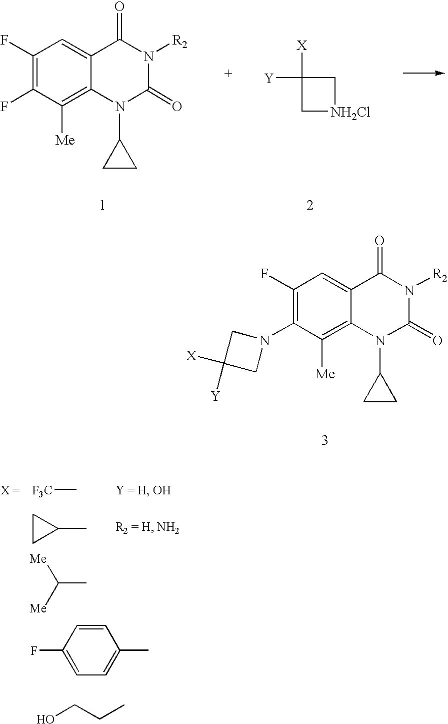

To a suspension of 3-amino-1-cyclopropyl-6,7-difluoro-8-methyl-1H-quinazoline-2,4-dione (1)(0.375 mmol) and the azetidine 2 (0.680 mmol) in 1 mL dimethylsulfoxide was added 0.25 mL triethylamine. The mixture was heated at 75° C. for 3 days. The reaction was cooled to 23° C. then poured onto 20% aqueous sodium hydrogensulfate and extracted with 3×10 mL with dichloromethane. The combined organic layers were dried over sodium sulfate, evaporated in vacuo, and purified by silica gel chromatography (gradient 1% to 15% isopropanol in dichloromethane) to give the target compound, typically as a white solid.

### Example B-2

### 3-Amino-1-cyclopropyl-7-(3-cyclopropyl-3-hydroxy-azetidin-1-yl)-6-fluoro-8-methyl-1H-quinazoline-2,4-dione (4)

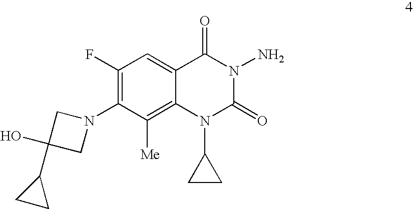

The title compound was prepared according to General Procedure B-1 as a white powder and was obtained in 28% yield. MS(APCI+): 361.1 ((m+1)/z).

### Example B-3

### 3-Amino-1-cyclopropyl-6-fluoro-7-[3-(4-fluoro-phenyl)-3-hydroxy-azetidin-1-yl]-8-methyl-1H-quinazoline-2,4-dione (5)

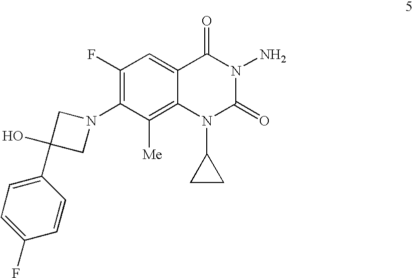

The title compound was prepared according to General Procedure B-1 as a white powder and was obtained in 18% yield. MS(APCI+): 415.0 ((m+1)/z).

### Example B-4

### 3-Amino-1-cyclopropyl-6-fluoro-7-[3-(2-hydroxy-ethyl)-azetidin-1-yl]-8-methyl-1H-quinazoline-2,4-dione (6)

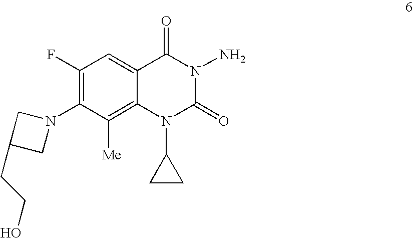

The title compound was prepared according to General Procedure B-1 as a white powder and was obtained in 34% yield. MS(APCI+): (349.1 (m+1)/z).

### Example B-5

### 3-Amino-1-cyclopropyl-6-fluoro-7-(3-hydroxy-3-isopropyl-azetidin-1-yl)-8-methyl-1H-quinazoline-2,4-dione (7)

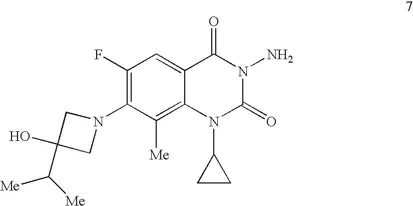

The title compound was prepared according to General Procedure B-I as a white powder and was obtained in 49% yield. MS(APCI+): 363.1 (m+1)/z.

### Example B-6

### 3-Amino-1-cyclopropyl-6-fluoro-7-(3-hydroxy-azetidin-1-yl)-8-methyl-1H-quinazoline-2,4-dione

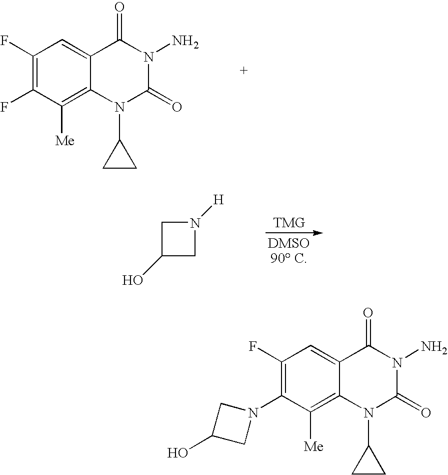

To a slurry of 3-amino-1-cyclopropyl-6,7-difluoro-8-methyl-1H-quinazoline-2,4-dione (0.60 g, 2.2 mmol) and 3-hydroxyazetidine (0.25 g, 3.4 mmol) in dimethyl sulfoxide (6 mL) was added 1,1,3,3-tetramethylguanidine (0.563 mL, 4.49 mmol), and the reaction mixture was heated at 90° C. for 18 hours. The mixture was cooled to room temperature, diluted with ethyl acetate (100 mL), washed with water (2×5 mL), brine (1×10 mL), dried over magnesium sulfate, filtered, and concentrated in vacuo. The resulting residue was purified on a 40M Biotage column using a methanol/dichloromethane solvent gradient to afford the title compound (0.45 g, 62%) as a white solid; mp=233-235° C.

### Example B-7

### 3-Amino-1-cyclopropyl-6-fluoro-7-(3-hydroxy-azetidin-1-yl)-8-methyl-1H-quinazoline-2,4-dione

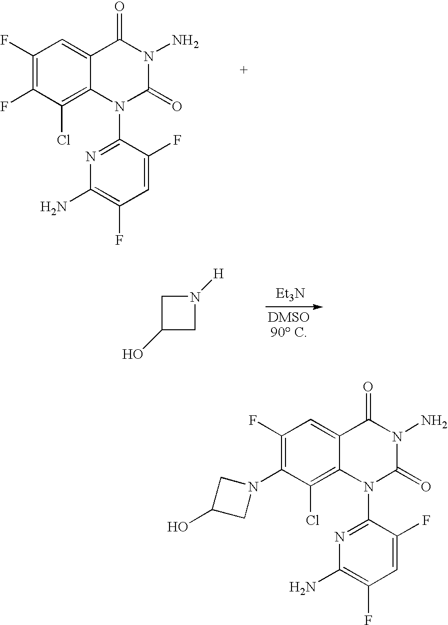

To a slurry of 3-amino-1-(6-amino-3,5-difluoro-pyridin-2-yl)-8-chloro-6,7-difluoro-1H-quinazoline-2,4-dione (0.30 g, 0.80 mmol) and 3-hydroxyazetidine (0.088 g, 1.2 mmol) in dimethyl sulfoxide (4 mL) was added triethylamine (1.1 mL, 8.0 mmol), and the reaction mixture was heated at 90° C. for 18 hours. The mixture was cooled to room temperature, diluted with ethyl acetate (100 mL), washed with 1N HCl (10 mL), brine (10 mL), dried over magnesium sulfate, filtered, and concentrated in vacuo. The resulting residue was triturated with a 1:1 dichloromethane:ethyl acetate solvent mixture and dried to afford the title compound (0.34 g, 99%) as an off-white solid; mp=212-214° C.

C. Formulations

The following illustrates representative pharmaceutical dosage forms, containing a compound of Formula I (“Invention Compound”), for therapeutic or prophylactic use in humans.

| (i) | Tablet | mg/tablet |
|---|---|---|
| | ‘Invention Compound’ | 25.0 |
| | Lactose | 50.0 |
| | Corn Starch (for mix) | 10.0 |
| | Corn Starch (paste) | 10.0 |
| | Magnesium Stearate (1%) | 3.0 |
| | | 300.0 |

The invention compound, lactose, and corn starch (for mix) are blended to uniformity. The corn starch (for paste) is suspended in 200 mL of water and heated with stirring to form a paste. The paste is used to granulate the mixed powders. The wet granules are passed through a No. 8 hand screen and dried at 80° C. The dry granules are lubricated with the 1% magnesium stearate and pressed into a tablet. Such tablets can be administered to a human from one to four times a day for treatment of pathogenic bacterial infections.

| (ii) | Tablet | mg/capsule |
|---|---|---|
| | ‘Invention Compound’ | 10.0 |
| | Colloidal Silicon Dioxide | 1.5 |
| | Lactose | 465.5 |
| | Pregelatinized Starch | 120.0 |
| | Magnesium Stearate (1%) | 3.0 |
| | | 600.0 |

| | Preparation for | | |
|---|---|---|---|
| (iii) | Oral Solution | Amount | |
| | ‘Invention Compound’ | 400 | mg |
| | Sorbitol Solution (70% N.F.) | 40 | mL |
| | Sodium Benzoate | 20 | mg |
| | Saccharin | 5 | mg |
| | Cherry Flavor | 20 | mg |
| | Distilled Water q.s. | 100 | mL |

The sorbitol solution is added to 40 mL of distilled water, and the invention compound is dissolved therein. The saccharin, sodium benzoate, flavor, and dye are added and dissolved. The volume is adjusted to 100 mL with distilled water. Each milliliter of syrup contains 4 mg of invention compound.

(iv) Parenteral Solution

In a solution of 700 mL of propylene glycol and 200 mL of water for injection is suspended 20 g of an invention compound. After suspension is complete, the pH is adjusted to 6.5 with 1 N hydrochloric acid, and the volume is made up to 1000 mL with water for injection. The Formulation is sterilized, filled into 5.0 mL ampoules each containing 2.0 mL, and sealed under nitrogen.

| (v) | Injection 1 (1 mg/mL) | Amount |
|---|---|---|
| | ‘Invention Compound’ | 1.0 |
| | Dibasic Sodium Phosphate | 12.0 |
| | Monobasic Sodium Phosphate | 0.7 |
| | Sodium Chloride | 4.5 |
| | N Sodium hydroxide solution | q.s. |
| | (pH adjustment to 7.0-7.5) | |
| | Water for injection | q.s. ad 1 mL |

| (vi) | Injection 2 (10 mg/mL) | Amount |
|---|---|---|
| | ‘Invention Compound’ | 10.0 |
| | Dibasic Sodium Phosphate | 1.1 |
| | Monobasic Sodium Phosphate | 0.3 |
| | Polyethylene glyco 400 | 200.0 |
| | N hydrochloric acid solution | q.s. |
| | (pH adjustment to 7.0-7.5) | |
| | Water for injection | q.s. ad 1 mL |

| (vii) | Injection 2 (10 mg/mL) | Amount |
|---|---|---|
| | ‘Invention Compound’ | 20.0 |
| | Oleic Acid | 10.0 |
| | Trichloromonofluoromethane | 5,000.0 |
| | Dichlorodifluoromethane | 10,000.0 |
| | Dichlorotetrafluoroethane | 5,000.0. |

All patents, and patent documents are incorporated by reference herein, as though individually incorporated by reference. The invention and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the spirit or scope of the present invention as set forth in the claims. To particularly point out and distinctly claim the subject matter regarded as invention, the following claims conclude this specification.

1. A compound of formula I 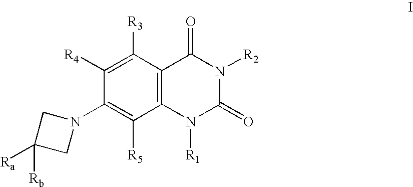
or a pharmaceutically acceptable salt thereof wherein
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is H,
NH₂, 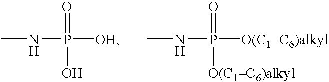
NH(C₁-C₆)alkyl,
NH(C₃-C₆)cycloalkyl,
NH-heteroaryl,
NHSO₂—(C₁-C₆)alkyl,
NHSO₂-aryl,
NHSO₂-heteroaryl, 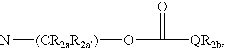
wherein, Q is O or is absent, and R₂ₐ and R_{2a′} are each independently H or (C₁-C₆)alkyl, or taken together with the carbons to which they are attached form a 3, 4, 5, or 6-membered substituted or unsubstituted ring, and R_{2b} is (C₁-C₆)alkyl, aryl, or heteroaryl, 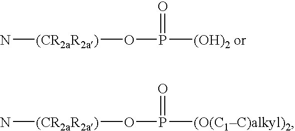
wherein R₂ₐ and R_{2a′} are as defined above, 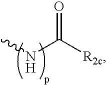
wherein 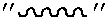
indicates the point of attachment, p is 0 or 1, and
R_{2c} is H,
(C₁-C₆)alkyl,
(C₃-C₇)cycloalkyl,
aryl,
heterocyclo,
heteroaryl, or 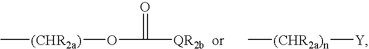
wherein R₂ₐ, R_{2b}, and Q, are as defined above, n is an integer from 0 to 10, and Y is OH, OPO(OH)₂, OPO(O(C₁-C₆))₂, or NR_{2d}R₂ₑ, wherein R_{2d} and R₂ₑ are each independently H, (C₁-C₆)alkyl, or (C₃-C₇)cycloalkyl, 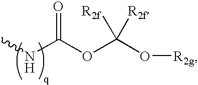
wherein q is 0 or 1, R_{2f} and R_{2f} are each independently H, (C₁-C₆)alkyl, aryl, or heteroaryl, or taken together with the carbon to which they are attached form a 3, 4, 5, or 6 membered ring, and R_{2g} is
(C₁-C₆)alkyl,
(C₃-C₇)cycloalkyl,
aryl, or
heterocyclo, or
heteroaryl;
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
Rₐ is H,
aryl,
(C₁-C₆)alkyl,
halo(C₁-C₆)alkyl,
(C₃-C₆)cycloalkyl, 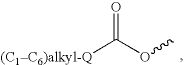
wherein 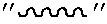
indicates the point of attachment and Q is O or is absent, 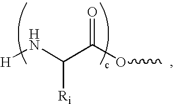
wherein 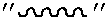
indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 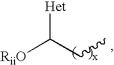
wherein 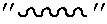
indicates the point of attachment, het is a 5- or 6-membered heterocyclo or heteroaryl group, and x is an integer of from 0 to 10; 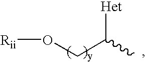
wherein 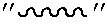
indicates the point of attachment, het is as defined above, and y is an integer of from 1 to 10;
wherein Rᵢᵢ is H,
(C₁-C₆)alkyl,
PO(OH)₂,
PO((C₁-C₆)alkyl)₂, 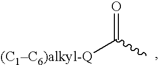
as defined above, or 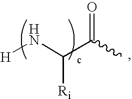
as defined above;
provided that 3 or fewer of R_{c} R_{d}, Rₑ, and R_{f} are H; or
R_{b} is OH,
OPO(OH)₂,
OPO((C₁-C₆)alkyl)₂ 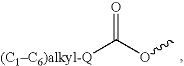
wherein 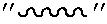
indicates the point of attachment and Q is O or is absent, 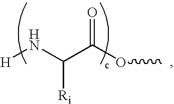
wherein 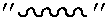
indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 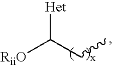
wherein 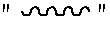
indicates the point of attachment, het is a 5- or 6-membered heterocyclo or heteroaryl group, and x is an integer of from 0 to 10; 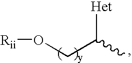
wherein 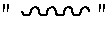
indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, 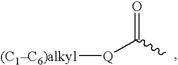
as defined above, or 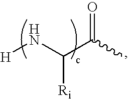
as defined above.

2. The compound of claim 1, wherein:
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is H,
NH₂,
NH(C₁-C₆)alkyl,
NH(C₃-C₆)cycloalkyl,
NH-heteroaryl,
NHSO₂—(C₁-C₆)alkyl,
NHSO₂-aryl,
NHSO₂-heteroaryl,
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
Rₐ is H,
aryl,
(C₁-C₆)alkyl,
halo(C₁-C₆)alkyl,
(C₃-C₆)cycloalkyl, 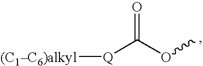
wherein 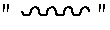
indicates the point of attachment and Q is O or is absent, 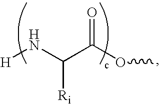
wherein 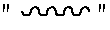
indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 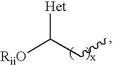
wherein 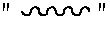
indicates the point of attachment, het is a 5- or 6-membered heterocyclo or heteroaryl group, and x is an integer of from 0 to 10; 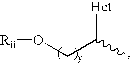
wherein 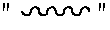
indicates the point of attachment, het is as defined above, and y is an integer of from 1 to 10;
wherein Rᵢᵢ is H,
(C₁-C₆)alkyl,
PO(OH)₂,
PO((C₁-C₆)alkyl)₂, 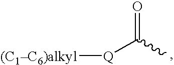
as defined above, or 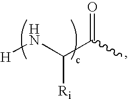
as defined above;
provided that 3 or fewer of R_{c} R_{d}, Rₑ, and R_{f} are H; or
R_{b} is OH,
OPO(OH)₂, 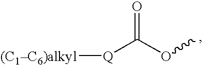
wherein 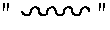
indicates the point of attachment and Q is O or is absent, 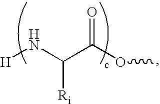
wherein 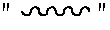
indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 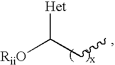
wherein 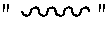
indicates the point of attachment, het is a 5- or 6-membered heterocyclo or heteroaryl group, and x is an integer of from 0 to 10; 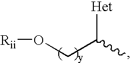
wherein 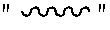
indicates the point of attachment, het is as defined above, and y is an integer of from 1 to 10;
wherein Rᵢᵢ is H,
(C₁-C₆)alkyl,
PO(OH)₂,
PO(O(C₁-C₆)alkyl)₂, 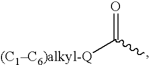
as defined above, or 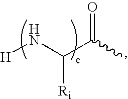
as defined above.

3. The compound of claim 1, wherein:
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is H,
NH₂,
NE(C₁-C₆)alkyl,
NH(C₃-C₆)cycloalkyl,
heteroaryl,
NHSO₂—(C₁-C₆)alkyl,
NHSO₂-aryl,
NHSO₂-heteroaryl,
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
Rₐ is H,
aryl,
(C₁-C₆)alkyl,
halo(C₁-C₆)alkyl,
(C₃-C₆)cycloalkyl, 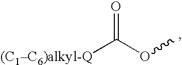
wherein 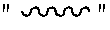
indicates the point of attachment and Q is O or is absent, 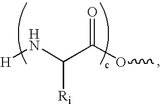
wherein 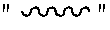
indicates the point of attachment, Rᵢ is H or (C₁-C₆)alkyl, and c is an integer having a value of from 1 to 10,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 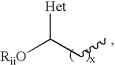
wherein 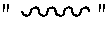
indicates the point of attachment, het is a 5- or 6-membered heterocyclo or heteroaryl group, and x is an integer of from 0 to 10; 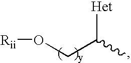
wherein 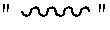
indicates the point of attachment, het is as defined above, and y is an integer of from 1 to 10;
wherein Rᵢᵢ is H,
(C₁-C₆)alkyl,
PO(OH)₂,
PO((C₁-C₆)alkyl)₂, 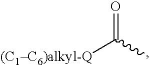
as defined above, or 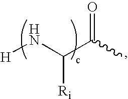
as defined above;
provided that 3 or fewer of R_{c} R_{d}, Rₑ, and R_{f} are H; or
R_{b} is OH,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 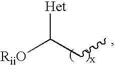
wherein 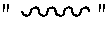
indicates the point of attachment, het is a 5- or 6-membered heterocyclo or heteroaryl group, and x is an integer of from 0 to 10; 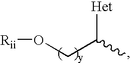
wherein 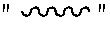
indicates the point of attachment, het is as defined above, and y is an integer of from 1 to 10;
wherein Rᵢᵢ is H or (C₁-C₆)alkyl.

4. The compound of claim 1, wherein:
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is H,
NH₂,
NH(C₁-C₆)alkyl,
NH(C₃-C₆)cycloalkyl,
NH-heteroaryl,
NHSO₂—(C₁C₆)alkyl,
NHSO₂-aryl,
NHSO₂-heteroaryl,
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
Rₐ is H,
aryl,
(C₁-C₆)alkyl,
halo(C₁-C₆)alkyl,
(C₃-C₆)cycloalkyl, 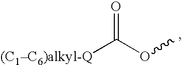
wherein 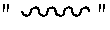
indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 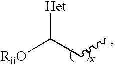
wherein 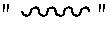
indicates the point of attachment, het is a 5- or 6-membered heterocyclo or heteroaryl group, and x is an integer of from 0 to 10; 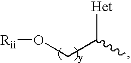
wherein 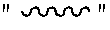
indicates the point of attachment, het is as defined above, and y is an integer of from 1 to 10;
wherein Rᵢᵢ is H,
(C₁-C₆)alkyl,
PO(OH)₂,
PO((C₁-C₆)alkyl)₂, 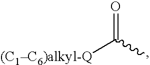
as defined above, or 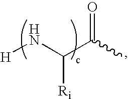
as defined above;
provided that 3 or fewer of R_{c} R_{d}, Rₑ, and R_{f} are H; or
R_{b} is OH,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 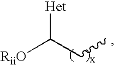
wherein 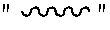
indicates the point of attachment, het is a 5- or 6-membered heterocyclo or heteroaryl group, and x is an integer of from 0 to 10; 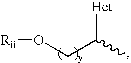
wherein 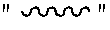
indicates the point of attachment, het is as defined above, and y is an integer of from 1 to 10;
wherein Rᵢᵢ is H or (C₁-C₆)alkyl.

5. The compound of claim 1, wherein:
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is H,
NH₂,
NH(C₁-C₆)alkyl,
NH(C₃-C₆)cycloalkyl,
NH-heteroaryl,
NHSO₂—(C₁-C₆)alkyl,
NHSO₂-aryl,
NHSO₂-heteroaryl,
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
Rₐ is H,
aryl,
(C₁-C₆)alkyl,
halo(C₁-C₆)alkyl,
(C₃-C₆)cycloalkyl, 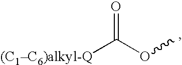
wherein 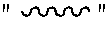
indicates the point of attachment and Q is O or is absent,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalky-O—,
RᵢᵢO(C₃-C₆)cycloalkyl-O—, 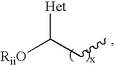
wherein 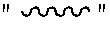
indicates the point of attachment, het is a 5- or 6-membered heterocyclo or heteroaryl group, and x is an integer of from 0 to 10; 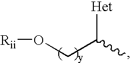
wherein 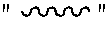
indicates the point of attachment, het is as defined above, and y is an integer of from 1 to 10;
wherein Rᵢᵢ is H, (C₁-C₆)alkyl, 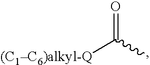
as defined above, or 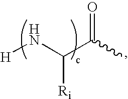
as defined above;
provided that 3 or fewer of R_{c} R_{d}, Rₑ, and R_{f} are H; or
R_{b} is OH,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—,
RᵢᵢO(C₃-C₆)cycloalkyl-O—, 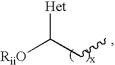
wherein 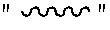
indicates the point of attachment, het is a 5- or 6-membered heterocyclo or heteroaryl group, and x is an integer of from 0 to 10; 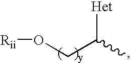
wherein 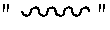
indicates the point of attachment, het is as defined above, and y is an integer of from 1 to 10;
wherein Rᵢᵢ is H or (C₁-C₆)alkyl.

6. The compound of claim 1, wherein:
R₁ is (C₁-C₆)alkyl,
halo(C₁-C₆)alkyl,
(C₃-C₆)cycloalkyl,
halo(C₃-C₆)cycloalkyl aryl, and
heteroaryl;
R₂ is H,
NH₂,
NH(C₁-C₆)alkyl,
NH(C₃-C₆)cycloalkyl,
NH-heteroaryl,
NHSO₂—(C₁-C₆)alkyl,
NHSO₂-aryl,
NHSO₂-heteroaryl,
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
Rₐ is H,
aryl,
(C₁-C₆)alkyl,
halo(C₁-C₆)alkyl,
(C₃-C₆)cycloalkyl,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—,
RᵢᵢO(C₁-C₆)haloalkyl-O—, or
RᵢᵢO(C₃-C₆)cycloalkyl-O—, wherein Rᵢᵢ is H or (C₁-C₆)alkyl; and
R_{b} is OH,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—, or
RᵢᵢO(C₁-C₆)haloalkyl-O—, wherein Rᵢᵢ is H or (C₁-C₆)alkyl.

7. The compound of claim 1, wherein:
R₁ is (C₁-C₆)alkyl,
halo(C₁-C₆)alkyl,
(C₃-C₆)cycloalkyl,
halo(C₃-C₆)cycloalkyl
aryl, and
heteroaryl;
R₂ is H,
NH₂,
NH(C₁-C₆)alkyl,
NH(C₃-C₆)cycloalkyl,
NH-heteroaryl,
NHSO₂—(C₁-C₆)alkyl,
NHSO₂-aryl,
NHSO₂-heteroaryl,
R₃, R₄, and R₅ are each independently H,
halo,
NH₂,
(C₁-C₆)alkyl,
halo(C₁-C₆)alkyl,
(C₁-C₆)alkoxy, or
halo(C₁-C₆)alkoxy;
Rₐ is H,
aryl,
(C₁-C₆)alkyl,
halo(C₁-C₆)alkyl,
(C₃-C₆)cycloalkyl,
RᵢᵢO(C₁-C₆)alkyl,
RᵢᵢO(C₁-C₆)haloalkyl,
RᵢᵢO(C₃-C₆)cycloalkyl, or
RᵢᵢO(C₃-C₆)cycloalkyl-O—, wherein Rᵢᵢ is H or (C₁-C₆)alkyl; and
R_{b} is OH,
RᵢᵢO(C₁-C₆)alkyl,
RᵢᵢO(C₁-C₆)haloalkyl,
RᵢᵢO(C₃-C₆)cycloalkyl,
RᵢᵢO(C₁-C₆)alkyl-O—, or
RᵢᵢO(C₁-C₆)haloalkyl-O—, wherein Rᵢᵢ is H or (C₁-C₆)alkyl.

8. The compound of claim 5, wherein
R₁ is (C₁-C₆)cycloalkyl and halo(C₁-C₆)cycloalkyl,
aryl, or
heteroaryl;
R₂ is H or NH₂;
R₃ is H or NH₂;
R₄ is H or halo; and
R₅ is halo,
methyl,
trifluoromethyl,
methoxy,
fluoromethoxy,
difluoromethoxy, or
trifluoromethoxy.

9. The compound of claim 5, wherein
R₁ is cyclopropyl,
fluorocyclopropyl, 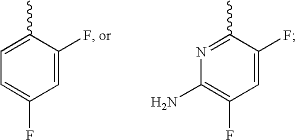
R₂ is H or NH₂;
R₃ is H or NH₂;
R₄ is H or F; and
R₅ is halo,
methyl,
trifluoromethyl, or
methoxy.

10. The compound of claim 2 wherein R₂ is H or NH₂, R₄ is H or F, and wherein A is 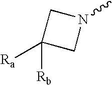
which is:

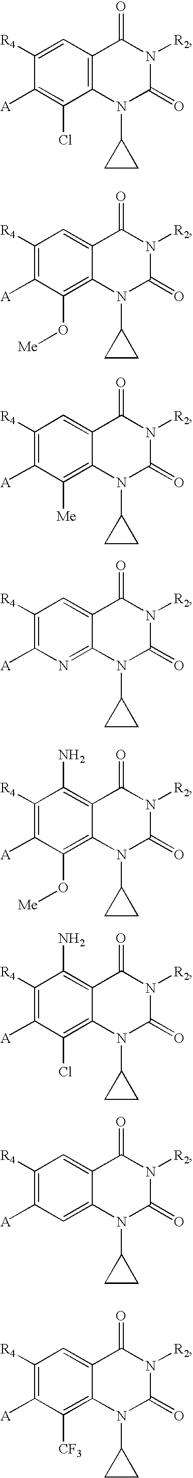

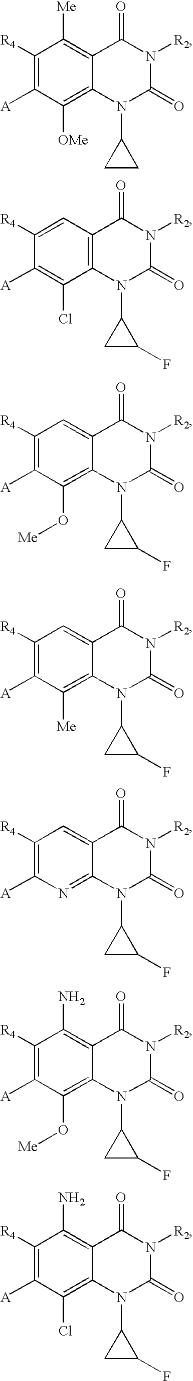

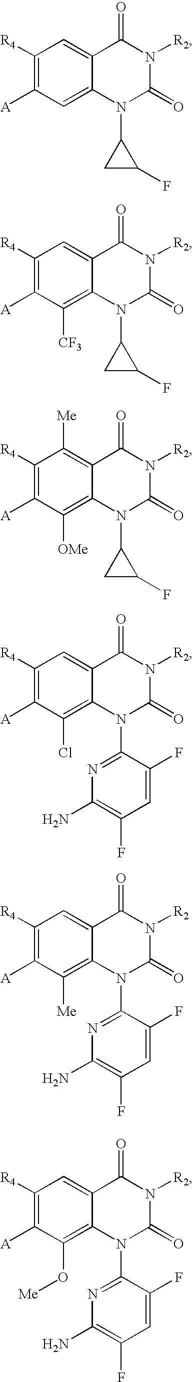

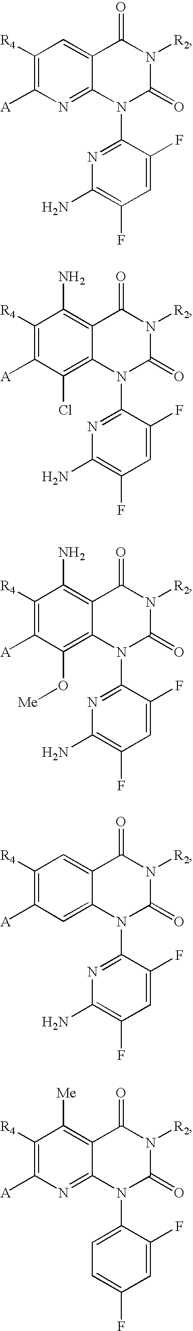

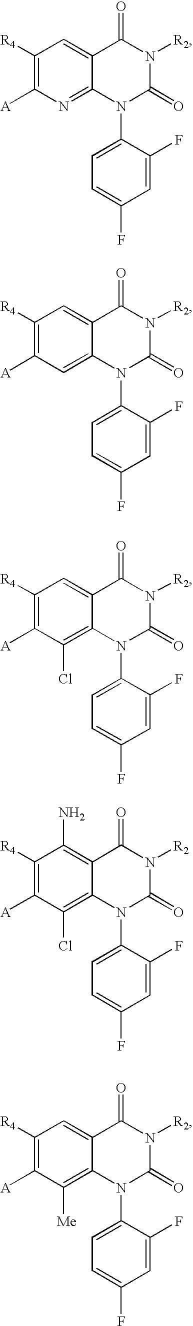

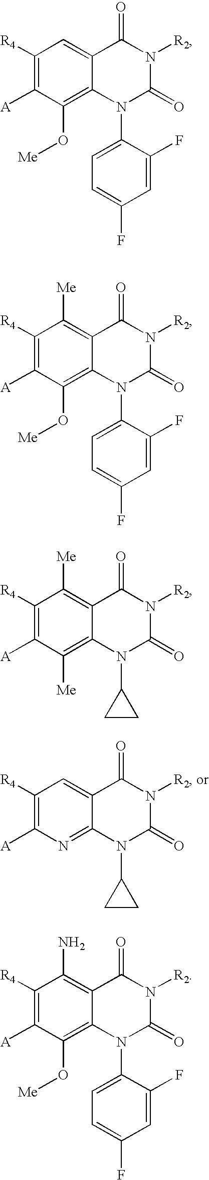

11. The compound of claim 2 wherein A is 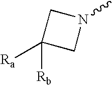
which is:

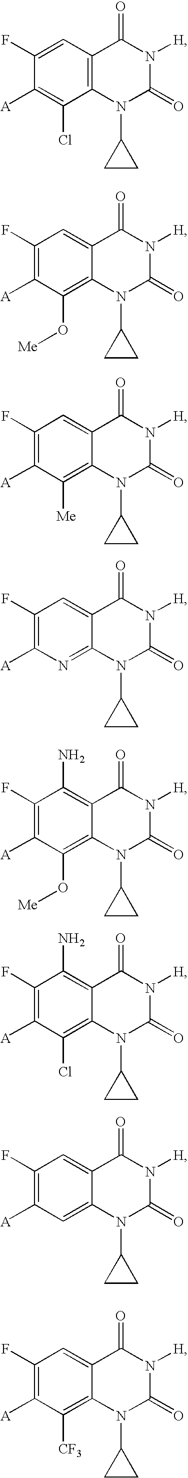

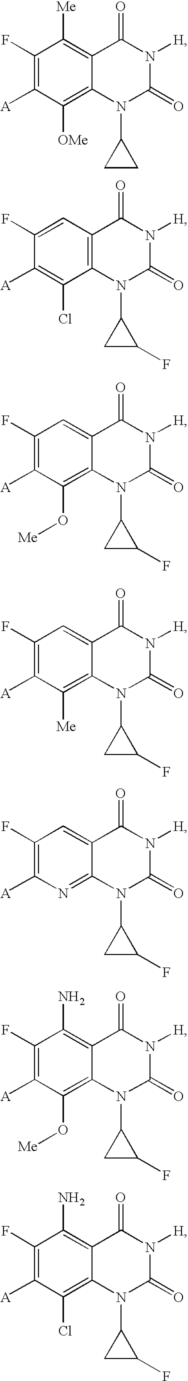

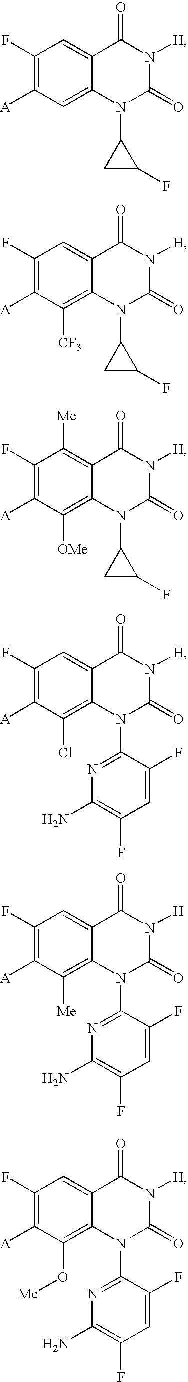

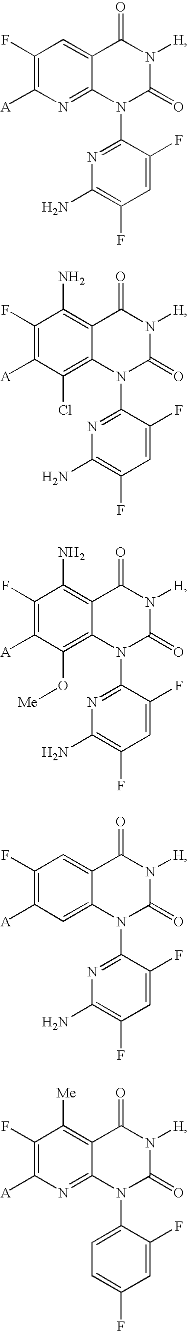

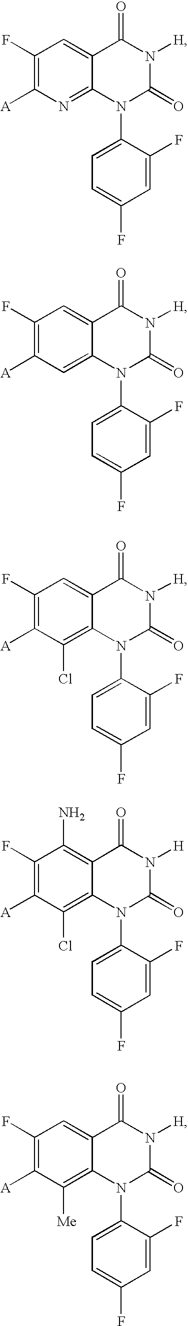

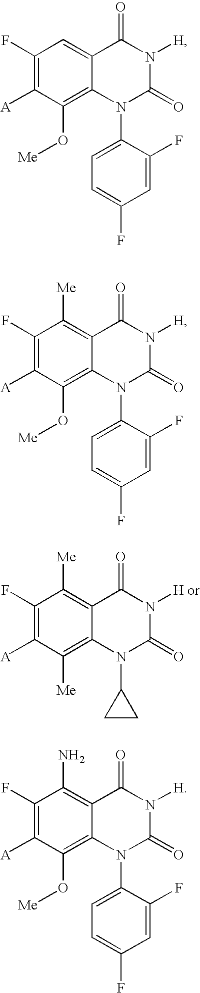

12. The compound of claim 4, wherein

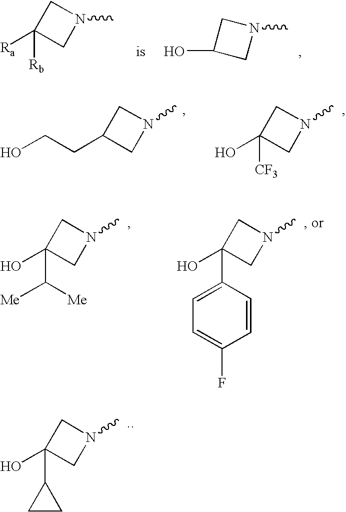

13. A compound which is
3-Amino-1-(6-amino-3,5-difluoro-pyridin-2-yl)-8-chloro-6-fluoro-7-(3-hydroxy-azetidin-1-yl)-1H-quinazoline-2,4-dione;
3-Amino-1-cyclopropyl-6-fluoro-7-(3-hydroxy-azetidin-1-yl)-8-methoxy-1H-quinazoline-2,4-dione;
1-Cyclopropyl-6-fluoro-7-(3-hydroxy-azetidin-1-yl)-8-methyl-4-oxo-1,4-dihydro-quinoline-3-carboxylic acid;
3-Amino-1-cyclopropyl-6-fluoro-7-[3-(2-hydroxy-ethyl)-azetidin-1-yl]-8-methyl-1H-quinoline-2,4-dione;
3-Amino-1-cyclopropyl-6-fluoro-7-(3-hydroxy-3-trifluoromethyl-azetidin-1-yl)-8-methyl-1H-quinazoline-2,4-dione;
3-Amino-8-chloro-1-cyclopropyl-6-fluoro-7-(3-hydroxy-3-isopropyl-azetidin-1-yl)-1H-quinazoline-2,4;dione
3-Amino-8-chloro-1-cyclopropyl-6-fluoro-7-[3-(4-fluoro-phenyl)-3-hydroxy-azetidin-1-yl]-1H-quinazoline-2,4-dione; or
3-Amino-8-chloro-1-cyclopropyl-7-(3-cyclopropyl-3-hydroxy-azetidin-1-yl)-6-fluoro-1H-quinazoline-2,4-dione.

14. A pharmaceutical formulation comprising a compound of claim 1 admixed with a pharmaceutically acceptable diluent, carrier, or excipient.

15. A method of treating a bacterial infection in a mammal, comprising administering to a mammal in need thereof an effective amount of a compound of claim 1.
